(19) United States
(12) Patent Application Publication
(10) Pub. No.: US 2023/0082362 A1
(43) Pub. Date: Mar. 16, 2023

(54) PROCESSES AND METHODS TO PREDICT BLOOD PRESSURE

(71) Applicant: NANOWEAR INC., Brooklyn, NY (US)

(72) Inventors: Vijay Varadan, State College, PA (US); Prashanth Shyam Kumar, Danville, PA (US); Mouli Ramasamy, State College, PA (US); Pratyush Rai, State College, PA (US); Venkatesh Varadan, Brooklyn, NY (US)

(73) Assignee: NANOWEAR INC., Brooklyn, NY (US)

(21) Appl. No.: 17/941,880

(22) Filed: Sep. 9, 2022

Related U.S. Application Data
(60) Provisional application No. 63/242,113, filed on Sep. 9, 2021.

(51) Int. Cl. A61B 5/00; A61B 5/021; A61B 5/0205

(52) U.S. Cl. CPC A61B 5/7275; A61B 5/746; A61B 5/7278; A61B 5/743; A61B 5/021; A61B 5/0205; A61B 5/7253; A61B 5/725; A61B 5/7267; A61B 5/7246

(57) ABSTRACT

The present invention relates to systems and methods to measure, compute, and predict blood pressure. More specifically, the invention generally relates to systems, methods, and process for predicting blood pressure from respiratory, circulatory, acoustic, hemodynamic, movement and blood flow characteristics and metrics.

### FIELD OF INVENTION

This application claims the benefit of U.S. Application No. 63/242,113 filed Sep. 9, 2021, the entire disclosure of which is hereby incorporated by reference.

The present invention relates to systems and methods to measure, compute, and predict blood pressure. More specifically, the invention generally relates to systems, methods, and process for predicting blood pressure from respiratory, circulatory, acoustic, hemodynamic, movement and blood flow characteristics and metrics.

### BACKGROUND

Hypertension is an important public-health challenge worldwide. In 2000, the estimated global number of adults with hypertension was 972 million, 26.4% of the adult population. Hypertension is important not only because of its high prevalence, but also because it is a major modifiable risk factor for cardiovascular and kidney disease. It was reported by WHO report 2002 that about 62% of strokes and 49% of heart attacks are caused by hypertension; 7 1 million die from hypertension, which is about 13% of the global fatality in total.

Nearly half of adults in the US (around 108 million) have hypertension, and only 1 in 4 adults have hypertension under control. In 2018, nearly half a million deaths in the US included hypertension as the primary or contributing cause. Similarly, high blood pressure costs the United States about $131 billion each year, averaged over 12 years from 2003 to 2014. The death rate from high blood pressure (BP) increased by nearly 11 percent in the United States between 2005 and 2015, and the actual number of deaths rose by almost 38 percent—up to nearly 79,000 by 2015.

Hypertension is a critical public-health challenge worldwide. Between 1990 and 2019, the number of men and women with hypertension doubled to 652 million and 626 million A pooled analysis of 1201 population-representative studies suggested that a dual approach of reducing hypertension prevalence through primary prevention, treatment, and control is achievable in the full spectrum of income settings. (Zhou, B. et al. Worldwide trends in hypertension prevalence and progress in treatment and control from 1990 to 2019: A pooled analysis of 1201 population-representative studies with 104 million participants, The Lancet 398, 957-980) Recently, Zhang et al. demonstrated the significance of intensive blood-pressure control in older Chinese patients resulting in a lower incidence of cardiovascular events. (Zhang, W. et al., Trial of intensive blood-pressure control in older patients with hypertension, N. Engl. J. Med., 385, 1268-1279).

The potential sources of errors in blood pressure measurement remain a significant challenge even in controlled clinical settings. Discrepancies in measurements as obtained by different operators raise the concern of methodologic errors by one operator, such as under-cuffing, excessive pressure on the head of the stethoscope, rapid deflation of the cuff, or use of different arms Kallioinen et al. investigated 29 potential sources of inaccuracy and categorized them as relating to the patient, device, procedure, or observer. They found significant directional effects with 27 sources with some inconsistency in terms of the direction of whether increase or decrease in measured blood pressure. Some of the significant sources of directional effects caused changes in the range of −23.6 to +33 mmHg systolic blood pressure and −14 to +23 mmHg diastolic blood pressure (Kallioinen, N., Hill, A., Horswill, M., Ward, H. & Watson, M., Sources of inaccuracy in the measurement of adult patients' resting blood pressure in clinical settings, J. Hypertens., 35, 421-441, (2017)) Apart from the inaccuracies of measurements leading to incorrect hypertensive classifications, psychophysiological changes in patients with conditions such as white coat hypertension can lead to a false positive diagnosis of hypertension. The recommended measurement of blood pressure for the diagnosis of hypertension across several international guidelines is out-of-office or at-home blood pressure measurement using ambulatory blood pressure monitoring (ABPM) and Home blood pressure monitoring (HBMP). The available evidence suggests that HBPM and ABPM present similar values and correlate with target-organ damage. (Kollias, A., Ntineri, A. & Stergiou, G. S., Association of night-time home blood pressure with night-time ambulatory blood pressure and target-organ damage: A systematic review and meta-analysis, J. Hypertens., 35, 442-452, (2017)) However, there are challenges with both HBPM and ABPM.

Regarding HBPM, although patients are trained on how to perform an at-home blood pressure self-measurement, the circumstances that must be created for repeatability of measurements are impractical for some patients, and compliance to these instructions is also not guaranteed. In the case of ABPM, the operation of the cuff during a measurement is perceptible to the patient and makes the measurements susceptible to a condition like white coat hypertension. Some patients must perform physical work such as construction that precludes the use of an ambulatory monitor, which requires the cessation of all movements during the oscillometric measurement time window. Furthermore, cuff-based ABMP is known to interfere with sleep. (Kwon, Y. et al., Blood pressure monitoring in sleep: Time to wake up. Blood Press, Monit., 25, 61-68, (2020)).Reports on sleep quality while using a cuffless blood pressure monitoring device (CLBPM) that is based on pulse wave analysis from a finger photoplethysmography sensor are scarce. The sleep quality was found to be comparable to ABPM. (Watanabe, N. et al., Development and validation of a novel cuff-less blood pressure monitoring device, JACC 2, 631-642, (2017).

All commercially available ABPM and HBPM devices are calibrated to measure brachial blood pressure, not central blood pressure. A systematic review of invasive validation studies on the accuracy of estimation of aortic systolic blood pressure using non-invasive devices could not draw specific conclusions. apaioannou, T. G. et al., Accuracy of commercial devices and methods for noninvasive estimation of aortic systolic blood pressure a systematic review and meta-analysis of invasive validation studies, J. Hypertens, 34, 1237-1248, (2016)). Clinically, such devices are not yet prevalent due to significant variability in the estimation of aortic-systolic blood pressure, and the superior invasive measurement methods are only indicated for patients suspected of having coronary artery disease. Laurent, S., Sharman, J. & Boutouyrie, P., Central versus peripheral blood pressure: Finding a solution, J. Hypertens, 34, 1497-1499, (2016)). The clinical evidence only supports a marginal difference. It is plausible that the incremental value of using central instead of brachial blood pressure is masked by the errors in measurement of ABPM and HBPM, which are not encountered with invasive central blood pressure measurements. Therefore, marginal superiority central blood pressure offers as a risk predictor of cardiovascular events may be accessible with brachial blood pressure by improving the calibration method of the brachial or peripheral blood pressure monitoring device. (Id.)

With the advent of automated CLBPM, observer-related sources of errors may be mitigated by eliminating the subjective intra- and inter-observer variance. Device-related errors cannot be eliminated with any of the reported CLBPM methods that use the pulse waveform characteristics such as Pulse Wave Decomposition Analysis (Hametner, B. & Wassertheurer, S., Pulse waveform analysis: Is it ready for prime time?, Curr. Hypertens. Rep., 19, 73 (2017)), Pulse Wave Transit Time, (Mukkamala, R. et al., Toward ubiquitous blood pressure monitoring via pulse transit time: Theory and practice, IEEE Trans. Biomed. Eng., 62, 1879-1901 (2015)) Pulse Arrival Time (Chen, W., Kobayashi, T., Ichikawa, S., Takeuchi, Y. & Togawa, T., Continuous estimation of systolic blood pressure using the pulse arrival time and intermittent calibration, Med. Biol. Eng. Compu., 38, 569-574,(2000)), and estimation of the pre-ejection period because these methods require calibration against the readings from a cuff based sphygmomanometer for each patient. There are known confounders that decorrelate the relationship between photoplethysmography (PPG)-based features and systolic blood pressure and diastolic blood pressure, such as changes in arterial stiffness (which increases with age as Elastin in the arteries is gradually replaced by less elastic Collagen), arterial wall viscosity, and assumptions on the radius of the arterial lumen (gender and BMI-related difference). None of the methods reported in the literature thus far have considered these demographic data to develop a generalized cuffless blood pressure estimation method. Studies that have reported the use of only centrally measured cardiovascular features for the prediction of blood pressure are scarce. Innovative use of ECG and deep learning to predict blood pressure was reported (Simjanoska, M., Gjoreski, M., Gams, M. & Madevska Bogdanova, A., Non-invasive blood pressure estimation from ECG using machine learning techniques Sensors, 18, 1160 (2018)). but did not meet the minimum criteria for accuracy as per the IEEE and ANSI standards.

Despite the risk people with hypertension may face, lack of awareness makes the situation difficult to control. The Joint National Committee 7th report (JNC 7) stated that the percentage of persons in whom hypertension is properly controlled (BP <140 mmHg/90 mmHg) is limited; more than 30% of the hypertensive populations are still unaware of their condition and are therefore not receiving treatment.

BP measured in a clinical setting by a trained physician using the auscultative technique with the mercury column has been used as the standard measurement for clinical diagnosis for over 100 years. It is, however, becoming increasingly clear that this reading is often inadequate or even misleading to represent a patient's true BP status. On the other hand, ambulatory BP measurement (ABPM) and home (or self) BP measurement (HBPM) are shown to be superior to clinic BP measurement (CBPM) in predicting cardiovascular mortality. Comparing to CBPM, ABPM, and HBPM have the following advantages: (1) eliminate the white-coat effect; (2) helpful to the assessment of clinic effects, drug effects, and work influence on BP; (3) better predict cardiovascular events and mortality; and (4) cost effective.

Therefore, in 2008, the American Heart Association (AHA), American Society of Hypertension (ASH), and Preventive Cardiovascular Nurses Association (PCNA) published a joint scientific statement that recommended using HBPM and further stated that HBPM should become a routine component of BP measurement in most patients with known or suspected hypertension.

Current devices employed for ABPM and HBPM are usually developed based on the oscillometric method, which must be used with an inflatable cuff during measurement. Those systems have several drawbacks which hinder their popularizing in the broad masses. One of the major problems is the employment of an inflatable cuff during the measurement. Patients find the cuff pressure is intolerable, particularly those with very high BP and who need frequently repeated readings; petechiae of the upper arm and bruising under the inflating cuff may occur; sleep disturbance is common.

Moreover, to have an accurate measurement, an appropriate cuff size must be selected according to the upper-arm circumference of users. Applying a cuff that is inappropriately small or large against the upper-arm circumference will contribute a substantially false elevation or reduction to the BP readings. Educating practitioners about appropriately sized cuffs for out-of-office BP measurement is necessary but increases the workload of the nurses.

Finally, the readings by conventional devices may be insufficient indicators for hypertension. Since only intermittent measurements of single snapshot readings are provided, current devices are incapable of recording the time varying BP or capturing the dynamic state of the cardiovascular system throughout the day. In addition, study of pathogenesis of hypertension reveals that the systolic hypertension is dependent on a series of changes in the vasculature, the most important of which is increased central arterial stiffness. Those signals are diffused by the relative imprecision in the techniques utilized by current devices.

In the past few years, there was an emerging interest in developing non-invasive BP measuring devices without an occluding cuff. Leading investigators in this field suggest that BP can be estimated indirectly from pulse transit time (PTT), which is a time period taken for the pulse wave to travel along the artery and arrive at the periphery, or features such as pulse arrival time (PAT), pulse wave velocity (PWV), pre-ejection period (PEP), etc., or their combinations. Models that relate BP, PTT, and other physiological features have been developed based on biological and mechanical properties of the cardiovascular system, e.g., elastic modulus, dimensions, and stiffness of the intervening vessels. Based on these models, systems that use electrocardiographic, photoplethysmographic, and/or phonocardiographic sensors have been proposed for the cuffless and continuous measurement of BP.

Cuffless BP measuring devices successfully release the users from the cuffs and are therefore more suitable to be implemented into the HBPM or ABPM systems, where frequent measurements are usually needed. When they are designed as wearable devices, e.g., a shirt or watch and/or integrated with furniture at home, e.g., a chair or bed for unobtrusive BP monitoring, or on-skin BP devices based on flexible-stretchable-printable electronics, the long-term and out-of-office monitoring becomes more comfortable and thus more attractive to the patients.

In addition, those devices have the great advantage of being not only capable of providing a snapshot of BP, but also potentially being usable for continuous BP monitoring. This special feature makes them superior to CBPM for the prompt identification of cardiovascular risk. Also, since signals (e.g., arterial stiffness) are implemented into the estimation model, the cuffless devices are potentially more capable of providing informative indication of the patient's health condition.

Nevertheless, since the physiology coefficients employed for BP estimation are subject-dependent, calibration is crucial to ensure the accuracy of the cuffless devices. A major challenge is to find a simple and accurate way to calibrate the device individually or estimate BP directly without a calibration procedure.

Since cuffless approaches have become important in hypertension research in recent years, a section of this standard is devoted to the assessment process. It is crucial for the clinicians and engineers to join efforts in establishing an evaluation standard. Although existing standards for evaluating sphygmomanometers were developed for devices with an occluding or inflatable cuff, parts of them are still applicable to the evaluation of cuffless devices. The experiences of these current standards need to be carefully appreciated during the development of the new standard.

In typical settings of wearable cuffless devices, bio signals such as biopotentials and body motion signals are acquired by wearable sensors attached to a patient's body (or on the skin) and sent to a nearby intermediate terminal for processing and/or relaying to a remote terminal. The wearable sensors may be equipped with a sensor of optical transmitter and detector, accelerometer, pulse meter, thermometer, pressure sensor, and galvanic skin reflex (GSR) electrodes to monitor the user's health conditions and/or movements. The signals collected from the sensors may also communicate with the personal server, which in turn connects to a mobile gateway for further signal processing and storage. Cellular communication capability may be added to expand service coverage to outdoors. Wireless body area networks (BSN) have great potential to be implemented into the settings.

The current standard of care does not include the use of a cuffless wearable device to continuously monitor blood pressure without intervention from the user. Some wrist blood pressure monitors may be accurate if used exactly as directed. Wrist or finger based blood pressure monitors are extremely sensitive to body position and minute movements. Accuracy of blood pressure measurement at wrist largely depends on the difference in height between the wrist and the heart because of the confounding effect of the hydrostatic pressure caused by the limb blood column. To get an accurate reading the arm and wrist must be at heart level. Even then, blood pressure measurements taken at the wrist are usually less accurate than those taken at the upper arm. Discrepancies in measurements as obtained by different operators or in different settings raise concern of the white-coat response or methodologic errors by one operator, such as under-cuffing, excessive pressure on the head of the stethoscope, rapid deflation of the cuff, or use of different arms. In treating hypertension in even the minimally obese patient, an adequate size cuff must be used for all blood pressure determinations. When blood pressure is determined with the patient in any but the standardized back-and-arm-supported seated position, the clinician should acknowledge the possibility that the position may alter the patient's classification. Similarly, the diagnosis and management of hypertension requires multiple measurements of blood pressure.

Physicians make therapeutic decisions with existing devices based on a spot measurement based blood pressure measurement. Non-invasive blood pressure monitoring is a critical part of the long-term management of hypertension as a risk factor for life-threatening events like Myocardial Infarctions, acute stroke. Accurate and timely monitoring of blood pressure is necessary for the management of hypertension, and this will have a significant impact on lowering the longitudinal risk of life-threatening diseases related to cardiovascular disorders and stroke. Hypertension is a well-known risk factor for life-threatening and irreversibly debilitating cardiovascular diseases whose prognosis include ST elevated myocardial infarction (STEMI) and acute Stroke.

High blood pressure is a leading cause of cardiovascular diseases. Although the diagnosis of high blood pressure appears to be straightforward, in clinical practice, misdiagnosis is not rare. For example, the phenomena of isolated office hypertension (high blood pressure when at the doctor's office despite a normal blood pressure out of the office) and masked hypertension (normal blood pressure in the doctor's office and high blood pressure out of the office) lead to frequent over- and under-diagnosis. Untreated high blood pressure can cause the structure of kidneys to change, leading to kidney failure. High blood pressure can affect the blood vessels, eyes and hormone levels. Over the years, those changes and conditions become irreversible. Similarly, dialysis patients experience occult or masked hypertension. It requires monitoring of blood pressure at various times of the day. Remote and at-home blood pressure monitoring could be a good tool for medication titration, or behavioral therapy. Blood pressure can also vary during surgical procedures or between pre and post-surgery.

### SUMMARY OF THE INVENTION

In the present invention, a novel approach is presented to estimate systolic and diastolic blood pressure that uses only centrally measured physiological data. The method takes as data input data obtained from available known sources, e.g. the multiparametric data inclusive of two channels of ECG- and thoracic impedance, heart sounds near the apex of the heart, activity, and posture captured by the FDA cleared SimpleSense™ device (510 (k) number K212160) (Nanowear Inc. Brooklyn, N.Y.) combined with demographics data (e.g., age, gender, height, and weight) which is added as a predictor so that the confounding effects encountered by pulse wave-based techniques can be mitigated. SirupleSense™ is a non-invasive, wearable, and portable medical device that uses cloth-based nanosensor technology (Rai, P. et al., Nano-bio-textile sensors with mobile wireless platform for wearable health monitoring of neurological and cardiovascular disorders, J. Dectrochem. Soc., 161, B3116-133150, (2013)) (FIG. 6). The garment was designed with an emphasis on case of wearing and takes between 20 and 30 seconds for most subjects to put on.

In certain embodiments of the present invention, the method includes 5 steps: obtaining input data, conditioning the input data, conducting feature extraction, conducting feature selection, and obtaining output.

Although there are always feature extraction and then feature selection steps in the process of the presentation, in certain methods when a time series regression neural network is used for blood pressure, the feature extraction and feature selection are not performed explicitly as separate steps but instead are implicit in the way a neural network is trained. The training process effects the neural network to learn the features that are relevant as part of the neural networks parameters. Parameters are any constants or choices for variables required to be made or assumed as part of applying a mathematical or computational method. For example:—neural networks require initial values of weights for a given architecture—is a parameter. Gaussian process methods require an initial choice of kernel function which is one of a list of available options like radial basis function, matern kernel etc.

The present invention predicts blood pressure from central physiological data, such as ECG, heart sound, thoracic impedance, posture, and activity, unlike the current standard of care that relies on the derivation of blood pressure based on pulse wave features from a peripheral artery like the radial artery. Certain aspects of the present invention use ECG and heart sound acquired simultaneously using e.g. nanosensor technology and a Microelectromechanical system or MEMs technology-based microphone that convert vibrations observed at the microphone input port into electrical signals, specifically converting vibrations caused by the heart sounds in the subaudible to audible range for the estimation of blood pressure in the present invention. In a specific example using a Nanowear® SimpleSense™ harness, the microphone is embedded in the Nanowear® SimpleSense™ harness. The ECG and heart sounds are digitized and captured by the Nanowear® SimpleSense™ electronics component Signal Acquisition Unit. Other methods of measuring heart sounds may involve belts, adhesive patches, or harnesses with embedded microphones, or electronic stethoscopes held in place by a user or caregiver. Due to the nature of the features used to derive blood pressure, the blood pressure trends derived from the measurements are more accurate for central or aortic blood pressure trending. The present invention provides a plurality of blood pressure measurements for a given time period, and an overall average blood pressure measurement with respect to time, which the physician can use to make therapeutic and diagnostic decisions, where the predicted blood pressure values can be shown e.g. by displaying a graph representing a time-wise trend of the plurality of blood pressure values or are displayed as numeric systolic and diastolic values. These predicted blood pressure values can be used to compare the previously measured or predicted blood pressure to provide an assessment of the increase or decrease of blood pressure over a period of time, along with providing notification or alarms in case of rapid changes in blood pressure. The continuous blood pressure predictions and measurements prevent false decisions caused e.g. by white coat hypertension or white coat syndrome. These predictions or measurements are not confounded by the position of the arm or from motion while the user is ambulatory. This is because these confounding factors are also measured and accounted for. Because of this approach, the artifacts arising from movements can be isolated or rejected as needed. Blood pressure varies over time (for example, beat to beat, minute to minute, hour to hour, day to day and visit to visit) but also based on the site of measurement on the body (for example, along the upper limb, from the radial artery to the aorta). Spatially dependent BP variability and the interaction between time and spatially dependent variability are still completely neglected by hypertension specialists. The described invention provides additional insights. about the blood pressure data on a more granular level and as well as overall average such as the long-term patterns in the variation of blood pressure, from instantaneous changes as a response to specific activities performed by the wearer or patient to changes over the course of a day, weeks or years, which may be useful as predictors or prognostic indicators for patients with chronic diseases. The patterns of variations of blood pressure at different regions of the body can be predictive of different pathways of disease progression. For example, variations in blood pressure in the extremities such as the foot may be early indicators of progression of diabetic neuropathy. The present invention also can be used to assess the trends in the blood pressure by physicians leading to more accurate and effective clinical decisions. This can directly impact clinical decision making with accurate blood pressure measurements. Similarly, it can significantly improve the quality of patient care while reducing the cost of care, such as patient rehabilitation outside of hospitals.

It is an object of the present invention to provide an assessment of blood pressure from central physiological data like ECG, heart sound, thoracic impedance, posture, and activity.

It is a further object of the present invention to provide a system capable of collecting data and/or of treating high blood pressure, wherein the data includes physiologic data such as ECG, heart sound, thoracic impedance, posture and activity, blood oxygen saturation, blood electrolyte levels for sodium, potassium, chloride, and calcium, pH of sweat, pH of blood, skin temperature, estimated core temperature from skin temperature or actual core temperature, environmental factors such as altitude, air pressure, ambient temperature, relative humidity, and level of UV exposure and estimation of urinary bladder fullness from impedance.

It is additionally an object of the present invention to provide a method of collecting and assessing data relating to blood pressure to be used for personalized patient care. It is a further object of the present invention for the method to include treatment of high blood pressure or low blood pressure based on the assessment of the data.

In accordance with an embodiment of the present invention, an integrated system for assessing blood pressure is provided.

In accordance with another embodiment of the present invention, an integrated system for assessing and treating an abnormally high or low blood pressure is provided. In certain embodiments of present invention, the system may detect, process and report various blood pressure related features.

In certain further embodiments of the present invention, the system may make treatment determinations based on these findings.

In certain embodiments of present invention, the integrated system may detect one or more physiological values from the blood pressure of the patient. In certain preferred embodiments, the data comprises physiologic data such as electrical activity of the heart, mechanical functions of the heart through heart sounds, general electrical properties of the chest and thorax such as conductivity and characteristic whether capacitive or inductive, ionic composition of blood and sweat such as concentration of sodium, calcium, and potassium, level of activity and movement. In accordance with additional embodiments of present invention, the integrated system may compare one or more detected physiological values to predetermined physiological values in order to obtain a comparison result in real time. The input data of the present invention may include physiological values which can be detected by one or more sensors and electronics. In accordance with a further preferred embodiment of present invention, the sensor(s) may be nanostructured working electrode (WE) and counter electrode (CE), and silver-silver chloride reference electrode (RE). In certain embodiments of present invention, the sensor(s) may be an array connected to an electronics module that acquires sensor signals, sends electrical stimuli and communicates wirelessly to mobile device at programmable time intervals.

In accordance with a further embodiment of present invention, the system may compute a model for blood pressure and to determine the frequency and magnitude of delivery of therapeutics. In accordance with another embodiment of the invention, the system may include components to treat high or low blood pressure, sensing components for sensing one or more values of one or more physiological data of blood pressure, such that the sensing, analyzing and treatment of abnormal blood pressure is integrated.

In an embodiment of the present invention, a method for predicting, estimating, and/or displaying blood pressure is provided which comprises:
a. selectively collecting a plurality of input data from one or more measurement devices, selection and collection methods and/or processes;
b. conditioning the input data in step a using one or more data conditioning methods and processes: ;
c. conducting feature extraction on the conditioned data of step b using one or more feature extraction methods and processes to extract one or more features for blood pressure prediction;
d. conducting feature selection on the feature extracted data of step c using one or more feature selection methods and processes;
e. obtaining output which predicts, estimates and/or displays blood pressure from the feature selected data of step d by converting the feature selected data into predicted blood pressure values using one or more methods or processes selected from the group consisting of normalization, combination, transformation and combinations thereof. In step e, the output is converted selected features in step d into systolic and diastolic blood pressure values using a plurality of normalization, combination, and transformation methods and processes. These last conversion steps are model training and selection steps and are not related to feature engineering.

In certain embodiments, the input data is selected from the group consisting of input data and/or derivatives of input data. (Derivatives here mean the same as feature extraction where specific characteristics of the input data is calculated algorithmically from the unmodified raw input data. For example:—electrical activity-based measurements such as ECG (electrocardiogram) have patterns that are characteristic and relate to certain underlying physiological phenomena observed in patients that are relevant to blood pressure). More concretely, heart health can be assessed by extracting various waveform characteristics from the ECG such as QT waveform intervals, heart rate variability based on the change in the time interval between successive R peak waveforms whose changes indicate changes in physiology for the patient that may be relevant to blood pressure) from electrical activity based metrics, input data and/or derivatives of input data from bioimpedance based metrics, input data and/or derivatives of input data from mechanical action metrics including goniometric measurements of all joint angles, absolute inclination, and orientation of different parts of the body in multiple axes, photoplethysmographic measures at various sites on the body such as fingers, toes, ankle, foot, arms, thorax, neck, and forehead, input data and/or derivatives of input data from sounds including heart sounds, lung sounds, gastrointestinal sounds, joint sounds, input data and/or derivatives of input data from blood oxygen levels, input data and/or derivatives of input data from skin or body temperatures measured at different locations of the body including extremities, thorax, abdomen, head, input data and/or derivatives from input data that can modulate pressure, and sweat biomarkers measured at various areas of the body such as lactate, pH, alcohol, nicotine, sodium, glucose, urea, chloride, discrete blood, interstitial fluid, cerebrospinal fluid (CSF) biomarker panels, metabolic panels, input data and/or derivatives of input data from geographic location and altitude metrics, input data and/or derivatives of input data from patient historic data and combinations thereof.

In certain embodiments of the present invention, the predicted blood pressure values are displayed in a graph representing a time-wise trend of the blood pressure values. In other embodiments of the present invention, the predicted blood pressure values are converted into a display of numeric systolic and diastolic values.

In an embodiment of the present invention, the predicted, estimated and/or displayed blood pressure values are compared with a previously measured or previously predicted blood pressure values to provide an assessment on the increase or decrease of blood pressure over a period of time. In certain embodiments, the predicted, estimated and/or displayed blood pressure values include the use of a parameter selected from the group consisting of peripheral vascular resistance of the patient, coronary resistance of the patient, arterial stiffness of the patient, aortic blood pressure of the patient, aortic blood pressure of the patient, left ventricular end diastolic pressures, pulmonary artery and venous pressures, measurements of chest circumference around the bottom of the sternum of the wearer or user for whom the blood pressure estimates are being calculated, and combinations thereof. In certain further embodiments, the previously measured values are input data available from other tests like echocardiographic imaging, measurements from a catheterization procedure, or prescription of specific types of medications that affect the blood vessels such as vasodilators or vasoconstrictors and combinations thereof. In further embodiments of the present invention, the method includes alarms or notifications in case of rapid blood pressure changes. In additional embodiments, the method for predicting, estimating, and/or displaying blood pressure provides a degree of a confidence for each prediction, estimation and/or display of blood pressure in a range of from about 75% to about 95%. In certain other embodiments, the method for predicting, estimating, and/or displaying blood pressure provides a degree of a confidence for each predicted outcome associated therewith, with each of the degrees of confidence based at least on predicted data or historical data regarding outcomes of the blood pressure prediction models on a plurality of patients. The numerical range for confidence may be a percentage difference relative to the mean estimated for a particular instance of estimation of blood pressure. Alternatively, the value may be expressed in units of blood pressure mm of Hg such as ±3 mm of Hg or percentage, such as a number between 0 and 100. In preferred embodiments of the present invention, the numerical range for confidence will be from about 75% to about 95%.

In certain embodiments, the feature extraction conducted is selected from the group consisting of rule-based extraction of waveform features from time series physiological data, using the output clusters of an unsupervised clustering method applied on input data at different granularities of time, applying the input data to a trained neural network for an alternative application such as image classification and using the output of a penultimate or earlier layer, using the output of the discriminator component of a generative adversarial network and combinations thereof.

In another embodiment of the present invention, a method is provided for defining and processing data to provide model inputs to a blood pressure prediction model comprising the steps of:

a) obtaining input data selected from the group consisting of input data and/or derivatives from electrical activity based metrics, data or derivatives from bioimpedance based metrics, input data and/or derivatives from mechanical action metrics including goniometric measurements of all joint angles, absolute inclination, and orientation of different parts of the body in multiple axes, photoplethysmographic measures at various sites on the body such as fingers, toes, ankle, foot, arms, thorax, neck, and forehead, input data and/or derivatives from sounds including heart sounds, lung sounds, gastrointestinal sounds, and joint sounds, input data and/or derivatives from blood oxygen levels, input data and/or derivatives from skin or body temperatures measured at different locations of the body including extremities, thorax, abdomen, and head, input data and/or derivatives from input data that can modulate pressure, and sweat biomarkers measured at various areas of the body such as lactate, pH, alcohol, nicotine, sodium, glucose, urea, chloride, discrete blood, interstitial fluid, cerebrospinal fluid (CSF) biomarker panels, and metabolic panels, input data and/or derivatives from geographic location and altitude metrics, input data and/or derivatives from patient historic data and combinations thereof;

b) conditioning the input data using a reversible transformation selected from the group consisting of filtering in time, frequency, wavelet, or other domains defined by a span of output of a convolutional neural network prior to a final layer which is a connected layer, so that the transformation does not remove any information from the data that is being transformed;

c) processing the conditioned input data of step b to obtain model inputs to the blood pressure prediction model using a method selected from the group consisting of converting the input data in time-series, subjecting the input data to feature extraction, computation, and transformation as predefined waveform patterns or time-varying quantities; processing the input data with unsupervised learning methods, processing the input data with regression-based methods, processing the input data with a trained neural network to achieve feature extraction, transforming the input data into an image or higher or lower dimensional space using reversible transformations and combinations thereof

d) inputting the processed data of step c as model inputs into the blood pressure prediction model.

In certain embodiments, the model inputs are defined and processed using one or more methods selected from the group consisting of signal conditioning, features engineering (inclusive of feature extraction and feature selection), transformations (inclusive of dimensionality increase, reduction or summarization using statistical measures such as mean, standard deviation, and variance) and combinations thereof, prior to use as training data. In certain embodiments of the invention, the conditioning of the input data using a reversible transformation is selected so that the transformation does not remove any information from the data that is being transformed.

In embodiments of the present invention, the method further comprises a signal and model assessment method comprising:
i) using a correction method to remove data collected by a measurement device used in a manner that does not produce the correct measurement, wherein the correction method accounts for data quality and confounders and is selected from the group consisting of thresholding techniques for level of movement, adaptive filtering techniques for remediation such as recursive least squares filtering and combinations thereof.
ii) feature engineering the data through an extraction process followed by a selection process comprising transformation and/or decomposition followed by feature selection.
iii) performing a process for the signal and model assessment to provide inputs for the blood pressure prediction model for improvements, conditioning, and correction, wherein the process is selected from the group consisting of a normalization process, a combination process, a transformation process and combinations thereof;
iv) performing a process selected from the group consisting of improvement methods and processes, correction methods and processes and combinations thereof to account for data quality and confounders;
v) performing data conditioning methods and processes for data conditioning and preparation; vi) performing feature extraction methods and processes to extract a plurality of features for signal and model assessment from a plurality of measurement devices and historic patient data obtained after step ii;
vii) performing normalization, combination, and transformation methods and processes for the signal and model assessment to provide inputs for the blood pressure prediction model for improvements, conditioning, and correction.

In certain embodiments, the normalization processes are selected from the group consisting of n standard score, student's t statistic, studentized residual, standardized moment, min-max feature scaling or combinations thereof, a combination process selected from the group consisting of support vector machines, linear regression, bagged trees, gradient boosting trees, extreme gradient boosting trees, Adaboost trees, random forests, k-nearest neighbors, gaussian process regression, or other kernel-based regression techniques, multilayer perception neural networks, recurrent neural networks, convolution neural networks and combinations thereof, and the transformation process is selected from the group consisting of scaling, weighted averaging, logistic regression probabilities and combinations thereof. In certain preferred embodiments, the method of normalization is min-max feature scaling. In certain preferred embodiments, the method of combination is gradient boosting trees. In certain preferred embodiments, the method of transformation is scaling.

In alternative embodiments, the method further comprises a method of personalization of the model. Signal and model assessment methods are used to improve models performance in terms of accuracy of predicting blood pressure, and conditioning, correction, and preparation of signals for the process of feature extraction. These methods are applicable specifically to the case where improvements in the accuracy of blood pressure predictions are sought for a specific individual referred to as the process of personalizing the models that predict blood pressure. In previously described embodiments, the intent of the signal and model assessment is to do post hoc adjustment to existing models that predict blood pressure and essentially fine tune them. In this embodiment, models are developed that are personalized, using data from only one patient. The method of personalization comprises:
a. performing one or more improvement, conditioning, and/or correction methods or processes to account for data quality and confounders.
b. performing data conditioning methods and processes for data conditioning and preparation of the data;
c. performing one or more feature extraction methods and processes to extract a plurality of features for signal and model assessment from one or more measurement devices and historic patient data;
d. performing one or more feature selection methods and processes for selecting features that are relevant to blood pressure;
d. performing normalization, combination and/or transformation methods and processes for the signal and model assessment to provide inputs for the blood pressure prediction model for improvements, conditioning, and correction.

In additional embodiments, the method further comprises a continuous improvement method comprising:
a. performing improvements, conditioning and/or correction methods and processes to account for data quality and confounders;
b. performing feature extraction methods and processes to extract a plurality of features for signal and model assessment from a plurality of measurement devices and historic patient data;
c. performing feature selection methods and processes for selecting features that are relevant to blood pressure;
d. performing one or more normalization, combination, and/or transformation methods or processes for the signal and model assessment to provide inputs for the blood pressure prediction model for improvements, conditioning, and correction.

In certain embodiments of the present invention, a pre-trained model (i.e. a pre-trained neural network) on a population is further trained with additional input data from an individual to generate a blood pressure prediction model that is unique for that individual. In preferred embodiments, the population is a large population comprising at least 50 patients. In certain preferred embodiments, the population comprises at least 80 patients. In preferred embodiments, at least 20% of the population has hypertension. In certain embodiments, additional data is obtained through a continuous blood pressure measurement technique for a time period extending from about a minute up to about forty five days by measuring the blood pressure through a known technique, wherein the blood pressures measured is used as a personalized training set for an individual to generate a blood pressure prediction model that is unique for an individual. In certain embodiments, the additional data is continued over the duration of in hospital observation inclusive of invasive monitoring of blood pressure. In certain embodiments, this duration can be from about 1 to about 30 days. In certain other embodiments, the period of monitoring may have a lower limit based on the minimum recording length needed to get a single measurement of blood pressure from the continuous measurement method (including invasive and non-invasive continuous measurement). In certain embodiments, this additional data is obtained by measuring the blood pressure through a known technique selected from the group consisting of an arterial line, catheter-based aortic line, blood pressures at each systole and diastole stage of heart beats in each of the chambers of the heart and major arteries such as pulmonary artery and vein, superior and inferior vena cava, and aorta, or non-invasively through a cuff-based blood pressure measurement device used on the upper arm or wrist, cuff-less devices that use pulse wave characteristics like Pulse Wave Transit Time, Pulse Decomposition Analysis, and pule tonometry data to estimate blood pressure. In certain preferred embodiments, the additional data is used as a personalized training set for an individual to generate a blood pressure prediction model that is unique for an individual.

In embodiments of the present invention, the method predicts a degree of a confidence for each of the predicted outcomes associated therewith, each of the degrees of confidence based at least on the predicted data or the historical data regarding signal and model assessment methods and processes on a plurality of patients. In certain further embodiments of the present invention, the method predicts a range of a confidence such as between 75% to 95% for each of the predicted outcomes associated therewith, each of the confidence values based at least on the predicted data or the historical data regarding signal and model assessment methods and processes on a plurality of patients.

In certain embodiments, a generative neural network that is trained using a different data set such as one trained to generate heart sounds from ECG signals, In further embodiments, the input data applied to the discriminator generates a set of features that is used to train another neural network or machine learning model to predict blood pressure (e.g. Method 5).

In certain embodiments of the present invention, a nanosensor is used to obtain certain of the input data for example a thermosensitive nanosensor. For example, a thermosensitive nanosensor can comprise a substrate sandwiched between the insulating layer and a conductive layer; vertically standing nanostructures attached to the substrate; a conductive material on top of the nanofiber surface; a thermosensitive hydrogel layer on top of the conductive layer; and a cover layer on top of the thermosensitive hydrogel to prevent loss of moisture and mechanical stress. Alternatively, the thermosensitive nanosensor can include a substrate having a plurality of vertically standing nanostructures attached thereto, the plurality of vertically standing nanostructure being covered with a conductive material to form conductive coated nanostructures; a thermosensitive hydrogel adjacent to the plurality of conductive coated nanostructures; and a cover layer on top of the thermosensitive hydrogel to prevent loss of moisture and mechanical stress. The substrate may include a fabric sandwiched between an insulating layer and conductive layer. Disclosure of such thermosensitive nanosensors is found e.g. in U.S. Publication No. 20210000417, incorporated by reference herein.

In certain embodiments, the input data are obtained from wearable remote electrophysiological monitoring system such as that disclosed in U.S. Publication No. 20160183835, incorporated by reference herein. Such a system may include a garment having at least one nanostructured, textile-integrated electrode attached thereto; a control module in electrical communication with the at least one nanostructured, textile-integrated sensor, and a remote computing system in communication with the control module. In other embodiments, the input data are obtained using a non-invasive, wearable and portable medical device for evaluation and monitoring blood pressure, such as the wearable textile-based harness including an adjustable elastic horizontal band and an adjustable elastic vertical band as disclosed in U.S. Publication 20210177335, the disclosures of which are incorporated by reference herein. In other embodiments, the input data can be obtained through a bandage system capable of collecting data such as the system disclosed in U.S. Application No. 63/174,721, the disclosures of which are incorporated herein. The system may detect one or more physiological values from the wound of the patient. The system may compare one or more detected physiological values to predetermined physiological values in order to obtain a comparison result in real time. The physiological values may be detected by one or more sensors and electronics. The sensors may be nanostructured working electrode (WE) and counter electrode (CE), and silver-silver chloride reference electrode (RE). The sensor(s) may be an array connected to the electronics module that acquires sensor signals, sends electrical stimuli and communicates wirelessly to mobile device at programmable time intervals. The system may compute a composite score for wound healing and to determine the frequency and magnitude of delivery of therapeutics. Suitable sensors for use in the present invention are described in U.S. patent application Ser. No. 16/916,843, the entire disclosures of which is hereby incorporated by reference. Reference is also made to U.S. Patent Application 63/245,477 filed Sep. 17, 2021, the disclosure of which is hereby incorporated by reference.

In certain embodiments of the invention, physical and chemical measurements of interest are also obtained by the above methods to e.g. monitor the heart activity, monitor or assist wound healing or other useful purpose. For example, the system and method of the present invention could be used in association with a wearable remote electrophysiological monitoring system which includes a fully wearable textile integrated real-time ECG acquisition system with wireless transmission of data for the continuous monitoring of e.g. athletes during training and competition. In certain further embodiments, the system of the present invention can make treatment determinations based on the blood pressure findings, or when applicable, the other physiologic or chemical findings, alone or in combination with the blood pressure findings.

### BRIEF DESCRIPTION OF THE DRAWINGS

FIG. 1: Methods and processes to formulate prediction and computation model architectures to predict systolic and diastolic blood pressure.

FIG. 2: Exemplary implementation of an ensemble model that combines outputs from multiple models using a weighted sum.

FIG. 3: Exemplary multi-modal time synchronous features extracted from the data.

FIG. 4: Exemplary feature selection process for a systolic blood pressure prediction model using impurity-based feature importance using a random forest regression model.

FIGS. 5A and 5B: Exemplary evaluation of performance of a trained diastolic pressure prediction model. The predicted pressures are closely in alignment with the actual target measurements of blood pressure as seen in FIG. 5A. The corresponding errors in measurement are seen in FIG. 5B.

FIGS. 6A and 6B depict the SimpleSense™ device (6A) and the software platform as it would appear on a computer screen and a cell phone screen.

FIG. 7A shows the test set up for simultaneous SimpleSense™ recording and Blood Pressure readings from a Sphygmomanometer and FIG. 7B shows Placement of Omron Wrist cuff and SimpleSense™ for sequential measurement.

FIG. 8 is a graph showing cross signal features—ECG and heart sound.

FIGS. 9A-D are graphs showing subject population characteristics with 9A showing a histogram of age distribution, 9B showing the distribution of race, 9C showing a histogram of hypertension stratification and 9D showing distribution of body habitus (age vs. Body Mass Index (BMI)).

FIGS. 10A and 10B show distribution of sphygmomanometer (gold standard device) measurements with 10A showing systolic blood pressure and 10B showing diastolic blood pressure.

FIG. 11 shows the relative importance of features for predicting systolic blood pressure and diastolic blood pressure using impurity-based feature importance.

FIGS. 12A-12D are graphs showing comparison of actual and predicted measures of systolic blood pressures from the SimpleSense™-BP cuffless model and a baseline model that uses only demographics data. FIG. 12A is a scatter plot of predicted vs actual systolic blood pressure for test set only for SimpleSense™-BP device with the correlation coefficient. FIG. 12B is a Bland Altman plot for test set with limits of agreement and the mean and standard deviation of errors compared to the reference measurement. FIG. 12C is a scatter plot of predicted vs actual systolic blood pressure on test set only for baseline model. FIG. 12D is a Bland Altman plot on test set only with limits of agreement and the mean and standard deviation of errors compared to the reference measurement for the baseline model.

FIGS. 13A-13D show a comparison of actual and predicted measures of Diastolic blood pressures from the SimpleSense™-BP cuffless model and a baseline model that uses only demographics data. FIG. 13A shows a scatter plot of predicted vs actual systolic blood pressure for test set only for SimpleSense™-BP device with the correlation coefficient. FIG. 13B shows a Bland Altman plot for test set with limits of agreement and the mean and standard deviation of errors compared to the reference measurement. FIG. 13C shows a scatter plot of predicted vs actual systolic blood pressure on test set only for baseline model. FIG. 13D shows a Bland Altman plot on test set only with limits of agreement and the mean and standard deviation of errors compared to the reference measurement for the baseline model.

### DETAILED DESCRIPTION OF THE EMBODIMENTS

The present invention extracts features such as Time between R peak (ECG) and S1 (Heart sounds), the time between R peak (ECG) and S2 (Heart sounds), ECG waveform characteristics (P, Q, R, S and T segment amplitudes and times), the ratio of S1 and S2 RMS amplitudes (Heart sounds), S3 and S4 RMS amplitudes, the time between S1 and S2 (Heart sounds), R-R intervals (ECG), respiratory rate (thoracic impedance), posture and activity. These extracted features are obtained from central physiological parameters like ECG, heart sound, thoracic impedance, posture, and activity. The data source for the central physiological data can be obtained through use of measurement devices or patient historic data.

In the present application the term “features” refers to any quantities derived from input data.

Parameters are any values as part of machine learning models of neural network models that can be updated as part of the training process for these models. Hyperparameters are variables that need to be assigned or chosen for each type of machine learning or deep learning model that are specific to each type of model.

The present invention provides a plurality of blood pressure measurements for a given time period, and an overall average blood pressure measurement with respect to time, which the physician can use to make therapeutic and diagnostic decisions, where e.g., the predicted blood pressure values are shown by displaying a graph representing a time-wise trend of the plurality of blood pressure values or are displayed as numeric systolic and diastolic values. Confounding factors such as the position of the arm or motion while the user is ambulatory are also measured and accounted for. Because blood pressure can also vary based on the site of measurement on the body (for example, along the upper limb, from the radial artery to the aorta) and spatially dependent blood pressure variability and the interaction between time and spatially dependent variability are also measured and accounted for.

In certain preferred embodiments of the present invention, the method includes 5 steps: obtaining input data, conditioning the input data, conducting feature extraction, conducting feature selection, and obtaining output.

Input data can be obtained by known methods including patient medical history data such as past diagnoses, test results for blood biomarkers, proteins, metabolites, and cholesterol and non-invasive medical devices that are intended to be used for collecting biomedical vital signs such as ECG, photoplethysmography, heart sound, activity, and calculate heart rate and respiration rate.

The input data can be conditioned by e.g. filtering methods to remove undesired noise or patterns that are irrelevant to the estimation of blood pressure within the input data obtained. Filtering methods such as finite impulse response filters, infinite impulse response filters, recursive filters, wavelet denoising, signal smoothening using running averages or autoreressive moving averages, and adaptive filters, ideal filters and optimal filters. Conditioning can also include data preparation, wherein the format of the input data is transformed into a format compatible with the inputted features available for obtaining output (step 5). For example, some categorical data that can only take on pre-specified values such as previous heart attack history “Yes” or “No” can be coded as 1 for “Yes” and 0 for “No”.

Conditioning methods do not perform rejection of data based on potentially incorrect methods of acquiring input data. The input data can be corrected by e.g. rule based rejection of data that is known to be noisy or unusable for the blood pressure estimation due to the presence of noise that makes it impossible to observe the signals or in cases where data was captured under circumstances or conditions that would lead to bias in the information carried by the input that is correlated to blood pressure. Correction methods can also include an adaptive filtering method such as a recursive least squares filter that can differentially remove a noise with a known pattern while preserving the signal. An example of such a method is the use of accelerometer data that is reflective of movements, embedded in a wearable device, as a measurement of noise when it is known that movement specifically interferes with the measurement of ECG signals. A correction method can be used to remove data collected by a measurement device used in a manner that does not produce the correct measurement, wherein the correction method accounts for data quality and confounders and is selected from the group consisting of thresholding techniques for level of movement, adaptive filtering techniques for remediation such as recursive least squares filtering and combinations thereof. For example, if collecting data only when the patient or wearer of a medical device is stationary is a requirement for a particular measurement device, provided that the device possesses the means to measure movement, as present in Nanowear SimpleSense™ device by way of an accelerometer sensor, the data collected during movements can be corrected by rejecting or ignoring the data from further processing. Alternatively, remediation of the data by using the movement data to remove any patterns in the measurement that are caused solely due to movements. The correction method to account for the data quality and confounders such as thresholding techniques for level of movement, such as an amplitude threshold, frequency content threshold, or adaptive thresholds, and adaptive filtering techniques for remediation such as recursive least squares filtering.

There are multiple options for feature extraction based on the feature to be extracted. Feature extraction steps such as separate algorithms to obtain each type of feature that is relevant for blood pressure. The algorithms for extraction such as rule based search that define the feature mathematically in terms of its appearance in the input data such as a peak of specific amplitude and width of half-maximum specification used for duration of peak for peaks and intervals, and continuous wavelet transform for pattern recognition of ECG waveforms with a comparison against an existing set of templates for each of the ECG patterns, or for heart sounds a sequence of steps of applying mathematical operations such as wavelet transforms, signal averaging, and applying a rule to find the peaks of S1 and S2 based on thresholds on peaks and durations. alternatively, fuzzy or partially defined methods of extraction of features is possible through the use of neural networks that are trained for tasks that are different from the task of predicting blood pressure such as image classification. In this case, the method involves a domain transformation using neural networks to perform the transformation. The choice of neural network for transformation is made by selecting neural networks that were trained for image classification problems such as convolutional neural networks, re-encoding the input data into a format that is compatible with image input such as sequentially arranging the input data in a 2-dimensional matrix or stack of 2-dimensional matrices to form a 3-dimensional matrix with each dimension representing a primary color (red, blue, or green), and obtaining a set of features for each of the layers of the neural network but for the final output layer. All of these outputs are potentially relevant features that will be put through features selection in the following step to determining whether to include them as inputs for model training and selection to obtain output. The preferred methods are rule-based extraction of features with known and well-established physiological meaning for example:—the second heart sound S2 is known to have information correlated to blood pressure.

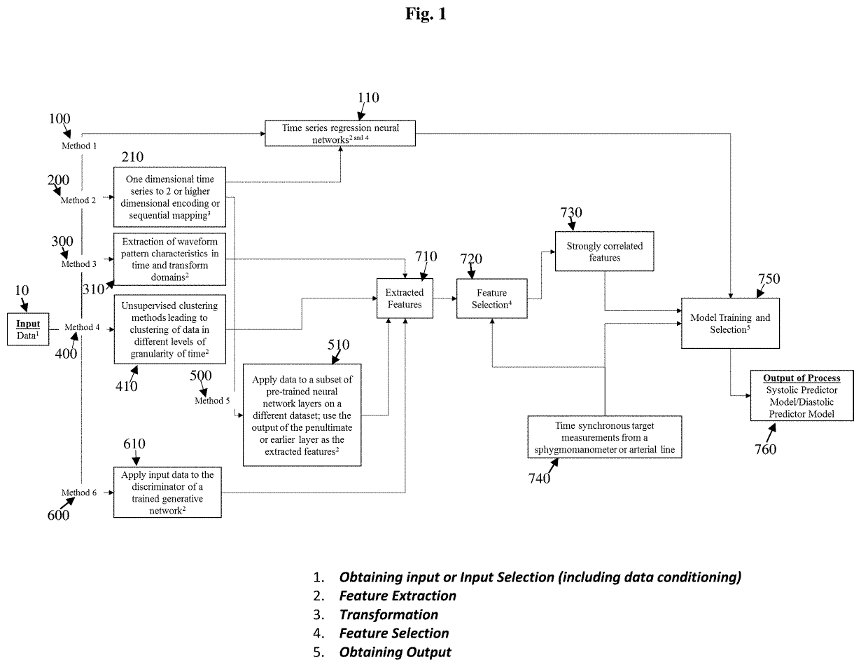

Feature selection is comprised of two steps. The first of these 2 steps is either transformation and/or decomposition and the second step is feature selection. Transformation and/or decomposition may not be performed always. It is required for certain methods such as Method 2 and 5 in FIG. 1. There are several methods and feature importance assessments for transformation and/or decomposition. Feature importance assessment provides a rank ordered list of features, with the top X number of features selected based on the accuracy obtained with a model trained with X number of features as input. Models can be exhaustively trained and the accuracy of models evaluate for all values of X from 1 to all features that were extracted. Transformation and/or decomposition of the features can be obtained by: e.g., applying Principle Component Analysis, eigen e or vector decomposition, and box cox transformations to perform transformations to the features. These methods are typically performed so that a large set of features can be compressed into fewer set of features that represent most if not all of the variations of the features in terms of pure information contained in the features as assessed by statistical variance (essentially, more variation means lore information). Feature selection evaluates whether the compressed form of information obtained through the transformation is relevant to blood pressure with the goal of selecting features that are relevant to blood pressure and eliminating or ignoring the others. Feature selection is done through the feature importance assessment which provides a rank ordered list of features based on how useful or relevant they are for the estimation of blood pressure. The top N number of features is then selected based on the accuracy obtained with a model trained with N number of features as input, allowing for exhaustive training and evaluation of accuracy of models for all values of N from 1 to all features that were extracted. Methods appropriate for selection can include measurement of mutual information using Kullback-Leibler converence, minimum redundancy maximum relevance, impish based feature importance using random forest regression models, F-statistic or f-test, neighborhood component analysis, backward elimination, forward selection, permutation feature importance, factor analysis, and relief algorithm for regression. Preferred methods are minimum redundancy maximum relevance and impurity-based feature importance using random forest regression models.

The output is obtained by applying an appropriate method to the selected features, such as support vector machines, linear regression, bagged trees, gradient boosting trees, extreme gradient boosting trees, Adaboost trees, random forests, k-nearest neighbors, gaussian process regression, or other kernel-based regression techniques, multilayer perception neural networks, recurrent neural networks, or convolution neural networks or combinations thereof. The preferred methods are a combination of gradient boosting trees, extreme gradient boosting trees, Adahoost trees, random forests, k-nearest neighbors, gaussian process regression, and support vector machines.

Although there are always feature extraction and then feature selection steps in the process of the present invention, in certain methods when a time series regression neutral network is used for blood pressure, the feature extraction and feature selection are not performed explicitly as separate steps but instead are implicit in the way a neural network is trained. The training process effects the neural network to learn the features that are relevant as part of the neural network parameters.

In other embodiments of the present invention, the overall procedure for prediction of blood pressures, systolic and diastolic, consists of 3 steps:

Step 1 is selecting and collecting a set of measurements (input data) that have information that is mutually shared with the predicted quantities, which are systolic and diastolic blood pressure. Input data is any data that is measured or available to begin the process of estimating blood pressure that is not processed or manipulated in anyway prior to using it for the estimation of blood pressure. It is selected solely based on its potential relevance to blood pressure estimation. (for example:—Electronic Medical Record (EMR) data inclusive of last known blood pressure or medication, ECG data collected at the time when blood pressure is estimated, or similarly heart sounds). Input data may also be time-series signals, i.e., signals measured periodically such as ECG, heart sounds, pulse oxygenation, pulse rate, activity levels, blood flow pulse wave velocity measured at different regions of the body such as wrist, foot, forearm, or near the heart, or measurements made with preserved information of time of measurements such as heart rate, blood biomarkers indicative of disease status, chronic diseases or metabolic abnormalities. In addition, data may include a patient's history and physical examination, information from insurance claims submitted by a patient that could be reflective of past medical problems or events, notes from electronic medical records, or patient-reported symptoms and notes, demographics data such as age, gender, height, and weight.

Step 2 is the conditioning and preparation of the input signals or discrete data points. Data conditioning is the process of removing, attenuating, or diminishing the presence of patterns in the physiologic data that are irrelevant to the physiological phenomenon being measured. These irrelevant patterns in the data are also referred to as noise. Data conditioning includes methods such as filtering, trend removal in case there are gradual drifts in the measurement values due to the instrumentation used to perform the measurement, or other signal processing methods that increase the proportion of physiologically relevant data to the noise.

Step 3 is feature engineering which is comprised e.g. of feature extraction and feature selection. In other words, feature engineering is the overall process and the sub processes are feature extraction (the derivation of features from the input data) and feature selection which is the process of choosing from the extracted features only those that are relevant to the estimation of blood pressure. Features are derived from the input data—they are summarizations or specific attributes of the input data that are deemed to be related to blood pressure.

Generally speaking, feature extraction is the process of measuring specific aspects within a physiological data set pertaining to a patient or summarizing the information present within a particular form of physiologic data. For example:—in an ECG physiologic waveform, there are characteristic patterns referred as Q wave and T wave. The time elapsed between the occurrence of a Q wave and a T wave is a feature that is relevant or related to blood pressure at the corresponding time of measurement of the ECG. The process of listing all such possible features and the developing computer programs and algorithms (Makowski, D., Pham, T., Lau, Z. J., Brammer, J. C., Lespinasse, F., Pham, H., Schölzel, C., & Chen, S. A. (2021), NeuroKit2: A Python toolbox for neurophysiological signal processing, Behavior Research Methods, 53(4), 1689-1696. that can calculate such features is known as feature extraction. This approach of feature extraction is applicable to statistical processes that are categorized under machine learning methods but are alternatives, or complementary to machine learning, where initial assumptions on the relationship between a particular feature and blood pressure are required. For example, a feature such as the time elapsed between the Q wave and T wave can be related to blood pressure, then a feature can be defined as QT interval, so there is an implicit assumption that QT interval is related to blood pressure for all patients which may not be true. Therefore, alternatively, the plurality of data as such without feature extraction and the accompanying required assumptions can be used as input to a neural network or deep neural network that is trained or optimized to estimate blood pressure directly from the input data in an approach that is commonly referred to as end-to-end machine learning where no features need to be pre-defined and no assumptions are made regarding which components or portions of the input data are relevant to blood pressure. The neural network training process will allow the neural network to infer the true relationship between the input data and blood pressure.

The process of feature extraction results in features and the process of feature selection results in a subset of features (these two steps together are referred to as feature engineering) that are actually relevant to blood pressure based on a measurement of the amount of mutual or shared information between the feature under evaluation (Mutual Information by definition relates two random variables. Mutual Information measures the dependence between the two from the information content perspective i.e., the measure of amount of information contained by a feature about the blood pressure.) The two random variables in this case are a particular feature and the blood pressure measured by a reference device such as a sphygmomanometer, simultaneously. and measures of blood pressure available from a reference device that is deemed as the ground truth measurement of blood pressure associated with the same time instance of the input data that was used to compute or extract the feature under evaluation. The method(s) may consist of a rule-based approach to measure specific attributes of an input data type such as the amplitude associated with a periodically occurring pattern in the input data such as the R peak in the ECG or the characteristic heart sound amplitudes in the measured heart sounds, S1 (lub) and S2 (dubb). The features to be used are based on domain expertise and known associations between the targeted features and blood pressure. Alternatively, an exhaustive evaluation of permutations of neural network architectures that may directly provide the transformation from the input data to features relevant for the estimation of blood pressure.

A preferred list of features important for systolic blood pressure are: Respiration rate 2, Relative Tidal Volume 1, Relative Tidal Volume 2, impedance, R to S1 time, R to S2 time, Mean interheat interval, the ratio of S1 RMS to S2 RMS. S tow, S mid, S high, QRS duration, age, height, and weight. A preferred list of features that are important for diastolic blood pressure are: Respiration rate 2, Relative Tidal Volume 1, Retati Volume 2, impedance, R to S2 time, Mean interheat interval, the ratio of S1 RMS to S2 RMS, S1 RMS, S low, S mid, S high, S1 low, age, height, and weight. These features may all be used in the present invention or used in groups and/or combined with additional features, as discussed below.

Data conditioning is the process of removing, attenuating, or diminishing the presence of patterns in the physiologic data that are irrelevant to the physiological phenomenon being measured. These irrelevant patterns in the data are also referred to as noise. The conditioning of data is done to essentially clean up the input data before feature engineering. The input data may contain known forms of noise or irrelevant information that it may be desirable to remove through conditioning before moving to feature engineering. Data conditioning includes methods such as filtering, trend removal in case there are gradual drifts in the measurement values due to the instrumentation used to perform the measurement, or other signal processing methods that increase the proportion of physiologically relevant data to the noise. The methods of conditioning of the input data may include, but are not limited to, the following:
1) transformations of the input data from the time domain to other domains such as frequency, wavelet using one of the wavelets (such as Coiflets, Daubechies, Meyer, Biorthogonal, Gaussian, Mexican hat, Morlet, Haar, Shannon, Complex Morlet) and/or cepstral analysis;
2) Application of filtering techniques such as homomorphic filtering, frequency-domain filtering of the time-domain, or transformed input signals to segment and extract quantitative or qualitative measures correlated with physiological factors in turn correlated to systolic and diastolic blood pressures. The determination of what to remove and what to retain is based on domain knowledge. For example, heart sound sensor or microphone recording the heart sounds on an individual may include conversation sounds from others nearby. These conversational sounds may be at a frequency range greater than the frequency range in which heart sounds manifest. A time-domain process filtering with the allowable frequency band set to the heart sound range can effectively remove the noise from the conversations of others in the vicinity.
3) Additionally, neural networks may be used to apply transformations. Data may also be encoded or embedded into a higher dimensional space that preserves or differentially enhances or improves the signal-to-noise ratio so that the shared or mutual information between the input data and blood pressures may be learned by a predictive machine learning model. This process is also referred to as basis expansion. An illustrative example is if the actual relationship between a measured input (X) and a value to be estimated (Y) in reality is A*X\hat{ }2+BX=Y, what is measured is only X, then higher dimensional embedding on X simply results in the input of X (actual measurements of X) and X\hat{ }A2 (which is the variation of X in another dimension that is not linearly related to X) as the higher dimensional embedding of X so that an estimation of A and B can be begun with this new transformed data consisting of X and X\hat{ }2) that preserves or differentially enhances or improves the signal-to-noise ratio so that the shared or mutual information between the input data and blood pressures may be learned by a predictive machine learning model.

The goal of data conditioning, with e.g. transformations or filters, is to remove to the maximal extent possible all variations within the data that are mathematically separable from the variations that are truly correlated to blood pressure. To accomplish this task, the data in its raw measured form may be subjected to transformations from the time domain to other domains such as frequency, wavelet using one of the wavelets (such as Coiflets, Daubechies, Meyer, Biorthogonal, Gaussian, Mexican hat, Morlet, Haar, Shannon, Complex Morlet), or cepstral analysis. As the next step to differentially enhance the information that is correlated to blood pressure relative to input data that is not correlated to blood pressure, filtering techniques may be applied such as homomorphic filtering, frequency-domain filtering of the time-domain, or transformed input signals to segment and algorithmically measure quantitative or qualitative measures correlated with physiological factors in turn correlated to systolic and diastolic blood pressures. Additionally, for the purpose of transforming the input data, neural networks may be used to apply transformations (All neural networks are generally a set of numerical computations consisting of multiplications, convolutions which is the same underlying computation as applying filtering in time domain, pairwise products known as dot or Hadamard products, or scaling (multiplying by a constant), or summation or applying various thresholding function to binarize or split into ranges any continuous number such as histograms, sigmoid functions, tan hyperbolic functions, Rectified linear unit (ReLU) or modifications thereof like leaky ReLU—these are also referred to as activation functions)—as the process of applying data to a neural network is essentially the same as applying a transfer function consisting of a sequence of mathematical operators (matrix multiplication, sum of all elements with or without multiplication by a scalar value referred to as weights, application of non-linear thresholding such as applying the input summation to a Logit function that limits the output between 0 and 1, and then applies a threshold for example 0.5 and all outputs above 0.5 are treated as 1 and below are treated as 0), that consist of a combination of mathematical operations and a selective transfer or ignoring of the output of parts of these computations referred to as skip connections). In the preferred embodiment, wavelet denoising, signal smoothening using running averages and Hilbert transform envelopes are used to condition the ECG and heart sounds data.

A pre-trained neural network is any neural network that is trained to perform either a classification (if the input is an image, the neural network output is whether there is a dog present in the image) or regression task (if the inputs are a geographic region and per capita income, then what is the estimate of house prices in that region). The pre-trained network is essentially not exposed to the input data chosen through the process of input selection described in this patent. However, the method or mechanism of extracting and emphasizing only certain features or aspects of the input to perform its function as trained originally, can be relevant to the estimation of blood pressure. Such a pretrained neural network, can then be provided with the training data prepared for blood pressure and can be trained or the internal terms or constants referred to as weights can be changed to better estimate blood pressure from the chosen inputs. The idea is that the pre-trained neural network will already possess a mechanism to infer attributes from any input data. By re-training the networks, the transfer function implemented by the neural network is fine-tuned so that it will predict blood pressure, instead of what it was originally trained to predict like whether there is a dog or not, and whether the region and per capita income is related to house prices in our example. Thus, the neural networks are not specifically designed for the purpose of predicting blood pressure, but can be used from a pre-trained network after replacing the input layer of the neural network to match the dimensions of the input data chosen in the previously in this step.

Preparation of data is the process of translating data in a certain format or data type into a format that is compatible with the next step which is the process of mathematically converting this data into values that are estimates of systolic and diastolic blood pressure. For example, when the mathematical conversion formula in the following step does not accept inputs in the form of text such as “Male” or “Female” and can only accept numbers or integers, then the data preparation step will perform the task of assigning numbers that represent “Male” and “Female” such as “Male” will be mapped or coded as 1 and “Female” will be mapped or coded as 2 prior to providing the data to the next step of mathematical conversion.

Preparation is optional because it only applies to situations where the input data is in a format that is not compatible with a computational model that is being evaluated. In contrast, data conditioning is always required. The preferred method (Method 3) does use data preparation as described in the “Male”/“Female” coding.

In a further step, a search is conducted for an optimal combination of the transformed or original data and a computational pipeline which is the sequence of computational operations needed from the input data to estimate blood pressure that includes data conditioning, preparation if needed, extraction of selected features and finally applying a computational model to estimate blood pressure using these features. The pipelines may include normalizations, combination, and transformation that converts the inputs into a systolic or diastolic blood pressure prediction that accurately matches simultaneously made ideal reference measurements from gold-standard devices such as sphygmomanometers or arterial line catheters.

The purpose of normalization is to standardize and restrict the range of the input data from reference sources that are used for model assessment and improvements such as sphygmomanometer readings, or arterial line readings. The purpose of combination is to have a method of determining the computational method needed to determine the extent to which the model and signals need to be improved for a model that is already estimating blood pressure. The transformation converts the outputs of the combination step to a value to improve or correct the model, or a signal that can improve or correct the signal, respectively.

The aforementioned steps could involve a plurality of techniques on how the data is used by a model, but more specifically following are some of the exemplary methods and techniques:

Input selection can be accomplished by gathering inputs from the human body such as, but not limited to, electrical activity (ECG, EMG, EOG, EEG), mechanical action (Goniometric measurements of all joint angles, absolute inclination, and orientation of different parts of the body in multiple axes, photoplethysmographic measures at various sites on the body such as fingers, toes, ankle, foot, arms, thorax, neck, and forehead), sounds (heart sounds, lung sounds, gastrointestinal sounds, and joint sounds), impedance (acoustic, electromagnetic, and ultrasonic), blood oxygen levels, temperatures measured at different locations of the body inclusive of extremities, thorax, abdomen, and head, sweat biomarkers measured at various areas of the body such as Lactate, pH, Alcohol, Nicotine, Sodium, Glucose, Urea, Chloride, discrete blood, interstitial fluid, cerebrospinal fluid (CSF) biomarker panels, and metabolic panels or any other features or metrics that can modulate blood pressure. In certain embodiments, the plurality of input data is selected from the group consisting of data or derivatives from electrical activity based metrics, data or derivatives from bioimpedance based metrics, data or derivatives from mechanical action metrics including goniometric measurements of all joint angles, absolute inclination, and orientation of different parts of the body in multiple axes, photoplethysmographic measures at various sites on the body such as fingers, toes, ankle, foot, arms, thorax, neck, and forehead, data or derivatives from sounds including heart sounds, lung sounds, gastrointestinal sounds, and joint sounds, data or derivatives from blood oxygen levels, data or derivatives from skin or body temperatures measured at different locations of the body including extremities, thorax, abdomen, and head, data or derivatives from features that can modulate pressure, and sweat biomarkers measured at various areas of the body such as Lactate, pH, Alcohol, Nicotine, Sodium, Glucose, Urea, Chloride, discrete blood, interstitial fluid, cerebrospinal fluid (CSF) biomarker panels, and metabolic panels, data or derivatives from geographic location and altitude metrics, data or derivatives from patient historic data and combinations thereof.

The use of the input data or measurement is to calibrate or tune the values estimated by blood pressure estimation mathematical formula to improve the accuracy for a specific individual. This is a means to personalize the blood pressure estimation method. Examples of such measurements or input data include most recent blood pressure measured by another device, most recent measurement of weight or height, and measurements from ultrasound echocardiograms or invasive catheterization procedures which can provide pressures in the chambers of the heart or in the major blood vessels. Personalization of the blood pressure estimation mathematical formula is only needed if it is observed that, for a specific individual, the estimation of blood pressure shows high errors of more than ±6 mm of Hg.

Feature selection may involve methods in two stages:

The first stage of feature engineering is feature extraction which involves techniques or methods such as, but not limited to, discrete Fourier and short-term Fourier transforms, discrete cosine transform, Autoregressive models (AR), Autoregressive moving average models (ARMA), classes of linear predictive coding models, cepstral analysis derived Mel-Frequency Cepstral Coefficients (MFCC), Kernel-based modeling, multiresolution analysis using discrete and continuous wavelet transformations, wavelet packet transformations and decompositions, empirical mode decompositions, power spectrum estimation using techniques such as Bartlett or Welch periodograms, Hilbert transforms, cross-spectral coherence that measures spectral coupling across different signal modalities, non-negative matrix factorization (NMF), ambiguity kernel functions, a subset of the layers from a pre-trained multilayer neural networks used as a transformation from input data into feature vectors in the feature space, unsupervised or supervised clustering methods like adaptive resonance-based neural networks, self-organizing maps, k-means clustering, k-nearest neighbors, Gaussian mixture models, and Naïve Bayes classifiers which group together similar feature sets (plurality of features extracted or plurality of statistically summarized inputs such as mean, standard deviation, or median) and assign group labels to each instance of a set of features. The preferred embodiment uses multiresolution analysis and signal decomposition using wavelet transforms to condition the heart sound data.

Supervised learning methods are applicable when a ground truth measurement of a quantity to be estimated is available, so the model can be trained to estimate that quantitative better during training. This is called “supervised” because the outputs generated by the model can be actually supervised and iteratively make changes to the model parameters to improve performance. Unsupervised methods on the other hand are more akin to data mining where measurements to guide the training process are not available. Instead, methods are used to define groups among instances of features that are more similar to each other than to instances of extracted features that are dissimilar. A cluster is defined as a group of points that are similar to each other, more so than other groups of points. A cluster classification is the identity given to a cluster. For example, if there are 2 distinct clusters, then cluster classification for a new feature set that falls within the group defined for group 1 is 1.

The second stage is feature selection which in turn involves two steps: 1) transformation and/or decomposition, and 2) selection. Transformation and/or decomposition is done to result in no change, or higher dimensionality of data, and the use of numerical methods to reduce dimensionality while preserving information and knowledge of variance. A transformation is the mathematical operation of manipulating a set of numbers that are representative of a state or measurement of a phenomenon using a series of mathematical operations. The manipulations may or may not be reversible in a mathematical sense. A reversible transformation is a mathematical method of manipulation of a data set that can be reversed or inverted and applied to the transformed data so that the original data set can be returned to. Such transformations are often used in filtering, where signal and noise are more easily separable by applying thresholds after being transformed. Wavelet denoising is a good example of this method—the transformation of input data in to the wavelet domain can be reversed or inverted so that the original data can be returned to without loss of information. Generally, transformations translate data from one domain to another. The present invention includes other domains to cover the use of neural networks to perform transformations like the use of a set of convolution neural networks with skip connections for example, which transform the data into an intermediate multi-dimensional space but not in any currently well-established definition of a domain (other than the traditional domains which are time frequency and wavelets).

Only reversible transformations are included for data conditioning. Decomposition is the process of reducing a set of data (simplistically a matrix of numbers and more generally a high dimensional data set) into a set of constituent fundamental sets of numbers that can represent all the information present in the original set of data. Principle component analysis is a form of decomposition of a given set of numbers into independent principal components that are equivalent in terms of the information present in the original matrix. Decompositions are useful to determine how many independent fundamental components are truly needed to represent all the information in a given set of data (fewer allows for reducing the amount of data needed to be stored and also complexity of the algorithms needed because less important components are, in a sense, discarded. Transformation and/or decomposition involves techniques like box cox transformation, eigenvalue, and vector decomposition, principal component analysis (PCA), kernel PCA, truncated singular value decomposition, multidimensional scaling, and isometric mapping, and t-distributed stochastic neighbor embedding. The purpose of transformation and/or decomposition is to reveal how much pure information is present within the set of features that were extracted. The relevance of the features is the evaluated during selection. Selection involves backward elimination, forward selection, random forests impurity-based importance, permutation feature importance, factor analysis, linear discriminant analysis, measurement of mutual information using kullback-leibler convergence, minimum redundancy maximum relevance, F-statistic or f-test, neighborhood component analysis, permutation feature importance, and relief algorithm for regression. Preferred methods are minimum redundancy maximum relevance and impurity-based feature importance using random forest regression models.

Features can be extracted for e.g. every 60 seconds of data from a nanosensor device. If a particular 60 second duration of recording is very noisy and the features cannot be extracted from that segment, then imputation techniques can interpolate and estimate what the value of the features might be for that 60 second segment based on historical values for the feature, most recently calculated feature values, or values in the neighboring vicinity in terms of time. Thus, in instances where features cannot be computed or are computed erroneously due to underlying noise in the input data, imputation techniques like replacement of missing values with mean, median, or interpolation from neighboring values with linear, or non-linear methods such as cubic splines or higher order polynomials may be used. Once missing values are imputed, regression techniques such as Tree-based methods, support vector machines, logistic and linear regression with variable coefficients, Gaussian process models, and neural networks. Tree-based methods may include but are not limited to boosted trees, random forests, gradient boosting trees, extreme gradient boosting, AdaBoost, bagged trees, and an ensemble of tree-based models, which is a combination of any or multiple tree-based models. Support vector machines may include but are not limited to linear, cubic, quadratic, coarse, fine, or medium gaussian. Gaussian process regression models may include but are not limited to rational quadratic, squared exponential, exponential, Matern 5/2 kernels. Neural networks may include but are not limited to multilayer perceptions, generalized regression neural networks, radial basis function networks, recurrent neural networks, long-short term memory networks, and convolution neural networks. The preferred embodiment is an ensemble average of regression models including k-nearest neighbor, Adaboost, gradient descent, support vector machines, and multilayer perceptions.

Based on the data types available as input, an appropriate model and specific methods can be chosen from the list above or other suitable methods. In practice, the different methods can easily be tested to determine which gives the best results with the particular input data. For example:—if the pulse transit time measurement is known, then simpler analytical models exist for the determination of blood pressure for the estimation of blood pressure using this data as input, such as described in (C. C. Y. Poon, et al., Modeling of Pulse Transit Time under the Effects of Hydrostatic Pressure for Cuffless Blood Pressure Measurements, 2006 3rd IEEE/EMBS International Summer School on Medical Devices and Biosensors, 2006, pp. 65-68, doi: 10.1109/ISSMDBS.2006.360098.) as

Blood pressure=K₁ * natural logarithm(pulse transit time)+K₂

Where K₁ and K₂ are subject specific parameters, so personalization could be further utilized to improve the performance of this model as described in an embodiment herein.

The systolic and diastolic prediction models are the output of a weighted average of a set of regression model outputs. The set of regression models may be at least one of a known set of regression models with architectures such as support vector machines, linear regression, bagged trees, gradient boosting trees, extreme gradient boosting trees, Adaboost trees, random forests, k-nearest neighbors, gaussian process regression, or other kernel-based regression techniques, multilayer perception neural networks, recurrent neural networks, or convolution neural networks. Bayesian optimization methods may be applied to the input features to determine the optimal set of hyperparameters for each of the models that make up the ensemble to achieve the least error between predicted and ground truth systolic and diastolic blood pressure values. Hyperparameter optimization may also be conducted using a semi-automatic or randomized grid search to explore all regions of the hyperparameter space for each of the regression models while trying to optimize the objective function, which is a means of measuring the error between the predictions and the ground truth measurements.

TABLE 1
List of preferred features used in preferred embodiments

| Name | Source | Description |
|---|---|---|
| Mean | Impedance | Mean respiration rate computed over a minute of thoracic |
| respiration | | impedance data from channel 1 from the three methods - sine |
| rate 1 | | fit, burg power spectrum and peak count. |
| Mean | Impedance | Mean respiration rate computed from thoracic impedance |
| respiration | | values measured over a period of time. |
| rate 2 | | |
| Relative Tidal | Impedance | Mean of the range of thoracic impedance values measured |
| Volume 1 | | over a period of time. |
| Mean thoracic | Impedance | Mean of the thoracic impedance values measured over a |
| impedance 1 | | period of time. |
| R to S1 time | Heart sound | Mean of the time intervals between the occurrence of the R |
| | and ECG | peak in the ECG and the S1 heart sound peak in the Heart |
| | | sound signal over a period of time. |
| R to S2 time | Heart sound | Mean of the time intervals between the occurrence of the R |
| | and ECG | peak in the ECG and the S2 heart sound peak in the Heart |
| | | sound signal over a period of time. |
| Mean | Heart sound | Average interval in seconds between heart beats. Both ECG |
| interbeat | | and Heart sounds are used to compute the mean heart rate. |
| interval | | |
| Ratio of S1 | Heart sound | Ratio of S1 rms and S2 rms |
| RMS to S2 | | |
| RMS | | |
| S1 RMS | Heart sound | Mean of the root mean square (RMS) amplitude over a 125- |
| | | millisecond window centered at the time of occurrence of the |
| | | S1 sound of the heart sound. A single value, the mean of the |
| | | RMS, is computed from over a period of time from the heart |
| | | sound signal. |
| S2 RMS | Heart sound | Mean of the root mean square (RMS) amplitude over a 125- |
| | | millisecond window centered at the time of occurrence of the |
| | | S2 sound of the heart sound. A single value, the mean of the |
| | | RMS, is computed from over a period of time from the heart |
| | | sound signal. |
| S | Heart Sound | Sum of amplitudes in the frequencies from 0 to 50 Hz relative |
| | | to total amplitude across all frequencies |
| S low | Heart sound | Sum of amplitudes in the frequencies from 50 to 100 Hz |
| | | relative to total amplitude across all frequencies |
| S mid | Heart sound | Sum of amplitudes in the frequencies from 125 to 175 Hz |
| | | relative to total amplitude across all frequencies |
| S high | Heart sound | Sum of amplitudes in the frequencies from 175 to 250 Hz |
| | | relative to total amplitude across all frequencies |
| S1 low | Heart sound | Sum of amplitudes in the frequencies from 50 to 100 Hz |
| | | relative to total amplitude across all frequencies for a sequence |
| | | of time period centered on the S1 peak sound |
| S1 mid | Heart sound | Sum of amplitudes in the frequencies from 125 to 175 Hz |
| | | relative to total amplitude across all frequencies for a sequence |
| | | of time period centered on the S1 peak sound |
| S1 high | Heart sound | Sum of amplitudes in the frequencies from 175 to 250 Hz |
| | | relative to total amplitude across all frequencies for a sequence |
| | | of time period centered on the S1 peak sound |
| S2 low | Heart sound | Sum of amplitudes in the frequencies from 50 to 100 Hz |
| | | relative to total amplitude across all frequencies for a sequence |
| | | of time period centered on the S2 peak sound |
| S2 mid | Heart sound | Sum of amplitudes in the frequencies from 125 to 175 Hz |
| | | relative to total amplitude across all frequencies for a sequence |
| | | of time period centered on the S2 peak sound |
| S2 high | Heart sound | Sum of amplitudes in the frequencies from 175 to 250 Hz |
| | | relative to total amplitude across all frequencies for a sequence |
| | | of time period centered on the S2 peak sound |
| QRS duration | ECG | Number of samples of the ECG data between the first zero |
| | | crossing before QRS complex and first zero crossing after the |
| | | QRS complex - denotes the demarcation of the QRS complex |
| Age | Demographics | Age of the subject |
| Height | Demographics | Height of the subject |
| Weight | Demographics | Weight of the subject |
| Gender | Demographics | Gender of the subject |

Table 1 lists preferred features for use in methods of the present invention. In preferred embodiments, all of the features of Table 1 are included. In a preferred embodiment, the method used to obtain a model is Method 3 of FIG. 1.

Although all of the features of Table 1 are preferably used in methods of the present invention, it is possible to use combinations of these features without including all of them. Additional features may also be used in combination with the features listed in Table 1 or as alternatives. Exemplary methods and processes to define 1 input data and how the input data is processed prior to use as training data for blood pressure prediction algorithms (i.e the methods to convert the input data into estimates of systolic and diastolic blood pressure) are found in FIG. 1. Potential choices of algorithms for use in these methods are elaborated on in the discussion of these methods below.

The computational model used to predict blood pressure can provide a level of confidence for the estimated blood pressure. In a concrete example, a Gaussian process regression model provides as an estimate of the mean and standard deviation of a gaussian distribution as the predicted blood pressure values. Therefore, with twice the standard deviation representing 95% likelihood (which is a characteristic of a Gaussian distribution) that the actual value of blood pressure falls within the predicted mean ±2 times the predicted standard deviation, a range of estimated blood pressure can be obtained within which the actual blood pressure will lie. For example—a confidence of estimated blood pressure may be ±5 mm of Hg if the predicted value is 170 mm of Hg as opposed to confidence of ±2 mmHg if predicted value is 130 mm Hg. Alternatively, this confidence of the estimate can be produced for this instance of blood pressure estimation as a cumulative effect of error estimates for each of the input data used for blood pressure estimation for this instance. For example:—if the feature defined as the peak amplitude of ECG waveform for all heart beats over a 10 second period of time is an input to blood pressure estimation and the standard deviation of these peak value measurements is treated as an error, then the confidence of blood pressure estimation can be proportional to the error of R peak amplitude measured. More variability will lower the confidence of the estimated blood pressure.

The computational method used for blood pressure estimation can be developed using a set of data collected from a patient population comprising at least about 50 patients, and preferably more than 80 patients. At least about 20% of the patients will have been diagnosed with a stage of hypertension, preferably distributed evenly across pre, stage 1 and stage 2 hypertension. Historically, the computational method used for blood pressure estimation was developed using a set of data collected from a patient population of at least 85 patients with at least 21 patients belonging to each of the stages of hypertension, and at least 50% of the patients are male (normal, pre-, stage1, and stage 2—hypertension. The criterion for categorization is as defined in Table 3). A computational model may be developed using the Method 3 by selecting the input data as all the data from Nanowear® SimpleSense™ device and demographic data such as age, gender, height, weight, and known category of hypertension among the groups normal, prehypertensive, stage 1, and stage 2 hypertension. The input data can be conditioned using finite impulse response filters and Butterworth filters for ECG, and wavelet denoising using ‘coiflet5’ wavelet for heart sounds. Signal averaging could further be used to reduce noise as part of signal conditioning. The features listed in Table 1 can be extracted using signal processing methods. For example:—algorithms defined to specifically discern patterns such as to find peaks in ECG waveform such as the R peak using an algorithm such as described by J. Pan et al.(J. Pan, W. J. Tompkins, “A Real-Time QRS Detection Algorithm,” in IEEE Transactions on Biomedical Engineering, vol. BME-32, no. 3, pp. 230-236, March 1985, doi: 10.1109/TBME.1985.325532.) and the time of its occurrence and similarly, peaks in the heart sound loudness such as the characteristic 51 and S2 heart sounds using an algorithm as described by Kumar P. S. et al. (Kumar, P. S., Rai, P., Ramasamy, M. et al., Multiparametric cloth-based wearable, SimpleSense, estimates blood pressure, Sci Rep 12, 13059 (2022). Once the features of table 1 are extracted, they are combined with the simultaneously acquired blood pressure measurements from a reference device such as a sphygmomanometer to create the training data set. After the creation of the trained data set, model training and selection is performed. Model training is the process of improving the accuracy of a model represented mathematically as a mathematical function or operation that associates a set of chosen inputs with the desired outcome that is predicted systolic and diastolic blood pressure. As each set of input data is presented, the model's mathematical calculation is performed to get an estimated output. This estimated output is compared to the ground truth value measured using a reference gold standard method, and an error also known as cost is determined using a pre-defined cost function. Some examples of cost functions are mean squared error, root mean squared error, mean absolute error, r squared, and log-loss. Depending on the value of cost determined by the difference between the estimated value and the expected value, the parameters or coefficients that are part of the model are updated following certain rules. Some examples of rules are gradient descent, root mean square propagation, adaptive resonance, and Adam optimizers. These methods are standard among neural network training Other methods in machine learning such as Support vector machines, Gaussian process regression, k nearest neighbors, Adaboost, extreme gradient descent all have algorithms for training that are unique to the methods and also require pre-specification of hyperparameters for the models when they are created (created essentially means defining a starting point or initialization point and a basic structure in terms of mathematical computational units used, some examples for gaussian process regression are choice of pre-defined kernel functions—5/2 matern, white noise, and radial basis function. Similarly for support vector machines some hyperparameters are—regularization parameter, kernel functions, and maximum iterations) for training. Computational models can be created using e.g. the six methods set out in FIG. 1, with Method 3 being the preferred method. The computational models provide a probabilistic estimate for the blood pressure given a set of inputs. The output can include a confidence interval (typically in mm Hg which is the same units as the estimated blood pressure) over which there is statistically high likelihood that the estimate matches the true value of blood pressure. Given the difference between the estimated blood pressure and the true blood pressure observed in the historical data used to develop the models, a confidence interval can be presented for current estimates of blood pressure computed by the models.

FIG. 1 shows six exemplative methods for formulating the prediction and computational model architectures to predict systolic and diastolic blood pressure. Each of these methods begins with the input of data as explained in Step 1 above.

Method 1 100 begins with a time series regression neural networks 110 performed on the input data 10. Generally, time-series data is a type of data that has a unique value associated with a series periodic or aperiodic instances of time. For example, stock ticker for AAPL has a series of traded share prices for each instance in time over the past year. The value of the stock over time is a form of time-series data. Time series regression neural network is a type of neural network that is used to mathematically search and determine the causal relationship between a sequence of events (like a sequence of data representative of the time varying ECG data or one of the input data components used for blood pressure estimation) and an output like the estimated blood pressure. They are specifically regression type networks because their output is continuously valued (i.e. can take the form of any continuous number—in this case the blood pressure value). Only an architecture is defined in this step (architectures are specifically defined by the number of layers, types of operations performed by the layers and how the output of a layer is transformed before applying it to the next layer, whether there are layers that are skipped and data from one layer forwarded to another layer further along in the architecture which can be viewed as a pipeline or stack, or certain outputs from one layer are discarded partially know.

After the time series regression neural networks is performed, a model training and selection is conducted 750 Model training is the process of improving the accuracy of a model represented mathematically as a mathematical function or operation that associates a set of chosen inputs with the desired outcome that is predicted systolic and diastolic blood pressure. As each set of input data is presented, the model's mathematical calculation is performed to get an estimated output. This estimated output is compared to the ground truth value measured using a reference gold standard method, and an error also known as cost is determined using a pre-defined cost function. Some examples of cost functions are mean squared error, root mean squared error, mean absolute error, r squared, and log-loss. Depending on the value of cost determined by the difference between the estimated value and the expected value, the parameters or coefficients that are part of the model are updated following certain rules. Some examples of rules are gradient descent, root mean square propagation, adaptive resonance, and adam optimizers. These methods are standard among neural network training Other methods in machine learning such as Support vector machines, Gaussian process regression, k nearest neighbors, adaboost, extreme gradient descent all have algorithms for training that are unique to the methods and also require pre-specification of hyperparameters for the models when they are created (created essentially means defining a starting point or initialization point and a basic structure in terms of mathematical computational units used, some examples for gaussian process regression are choice of pre-defined kernel functions—5/2 matern, white noise, and radial basis function. Similarly for support vector machines some hyperparameters are—regularization parameter, kernel functions, and maximum iterations) for training

In the Output of the Process, the Systolic Predictor Model/Diastolic Predictor Model 760 is performed. In preferred methods, several different types of models 750 are trained on the training data and their performance is evaluated on an independent test data set that is collected from a different group of subjects that the data from the training set. Depending on the error performance measured by established metrics such as mean squared error, mean absolute error, or r squared, the best performing model is selected. This is the selection part of 750. So, the output of 760 is the best performing model that includes all steps that led to the definition of that model inclusive of choice of input data, conditioning and preparation of data, transformation and/or decomposition of data, and finally the machine learning algorithm or combinations of machine learning algorithms that were used in the model. The inputs of Method 1 are applied to models after data conditioning which may involve filtering in time, frequency, wavelet, or other domains using a reversible transformation. Preferably, this involves filtering in time domain and wavelet domains. The choice of the data conditioning methods is dependent on the input data type and domain knowledge regarding what is a signal and what is noise or irrelevant information in the input data. For example frequency domain filtering is preferred when the input data is ECG and the noise source is power line interference. The inputs with or without conditioning may be retained in time-series format to be supplied to a class of neural networks or deep learning architectures that have output layers comprised of linear transformations that sum and scale the output from the previous layer commonly referred to as regression output layers, that support continuous-valued regression outputs (with deep learning architectures being a subset of neural networks that have several computational layers that categorizes them as deep, as opposed to shallow neural networks which have fewer computational layers). The output layer of a neural network produces a regression output, i.e., continuous valued output. The output layer is defined by either a single neuron in the case of separate models for systolic and diastolic blood pressures, or two neurons if systolic and diastolic pressures are estimated using separate models. Each neuron multiplies the weights (W1, W2, W3, and so on) assigned to each connection from the previous layers, sums the values, and finally applies a linear scaling or multiplicative factor to the sum to calculate an output. This output can be either systolic pressures, diastolic pressures, or both corresponding to the time window or time stamp of input data.

Data input needs to be conditioned if the data input has information that is not relevant to blood pressure. A specific example are heart sounds. All heart sound sensors are microphones, regardless of the underlying operating principles. The sensors will collect and record all sources of vibration that are seen at the sensing port of the microphone. However, the input data needed are only the heart sounds themselves, and not any stray noises or conversations occurring in the vicinity, or the wearer's own speech. Therefore, the heart sounds, which are data input, are conditioned using signal averaging and wavelet denoising. The conditioning is different for each type of data and the choice of conditioning method is an educated guess for those trained in digital signal processing methods.

It is of note that in Method 1, where a time series regression neural network is used for blood pressure estimation, feature extraction and feature selection are not explicitly performed by a programmer Instead, these steps are implicit in the way a neural network is trained. The training process effects the neural network to learn the features that are relevant as part of the neural networks parameters.

Method 2 200, one dimensional time series to 2 or higher dimensional encoding or sequential mapping 210 is performed on the input data 10. (Higher dimensional encoding, also known as basis expansion is discussed in detail above). Sequential mapping is the conversion of a data set that is multidimensional into a one-dimensional data set. For example, if the data input includes ECG data, heart sound data and altitude data having a different number of measured values or samples per second and represent different physical entities, these series of numbers can be sequentially appended with ECG first, then heart sounds, then altitude to get a one-dimensional array. This method of preparing input data is called sequential mapping.

After the one-dimensional time series to 2 or higher dimensional encoding or sequential mapping 210 is performed on the input data, Method 2 200 then proceeds 200 A to the steps of Method 1 100 above, with a time series regression neural networks performed on the output of 210, after which a model training and selection is conducted 750 and then the Output of the Process, the Systolic Predictor Model/Diastolic Predictor Model 760 is performed. In the third path 725C, strongly correlated features 730 are selected from the output of 720 and input into the Model Training and Selection 750. Simultaneously, Time synchronous target measurements from a sphygmomanometer or arterial line 740 are input directly into the Model Training and Selection 750. The step of using a calibration value is needed when there is a limitation on what data is available as input data. In some cases, the data available for blood pressure estimation may only be relevant to tracking changes in blood pressure rather than being able to estimate actual values of blood pressure. In such cases, a recent measurement of blood pressure will provide a starting point from which the available input data can estimate blood pressure with acceptable accuracy that is established for clinical requirements such as an error of no greater than 5 mm of Hg over about 255 or more measurements made by the device or model under evaluation. In the preferred embodiment, there is no need for calibration because demographics data, including age, height, weight, and gender is used to arrive at the most appropriate blood pressure estimate and then track variations using the listed inputs for changes in blood pressure.

Finally, the Output of the Process, in which the Systolic Predictor Model/Diastolic Predictor Model 760 is performed.

In Method 2 200, the input data may be transformed into an image or higher dimensional space using reversible transformations that do not result in any loss of information. Interpolation and serialization or encoding methodology may be used to convert the single-dimensional time series data into multidimensional data. When an image is specifically 2 dimensional data, higher dimensional data is higher in dimensionality compared to the input data, so it can be any number of dimensions as an upper limit. The dimensionality is practically limited by the computational hardware and the available memory. Once transformed, the higher dimensional data may be used to train convolution neural networks or one of a family of recurrent neural networks inclusive of but not limited to long short-term memory (LSTM) networks or bilateral-LSTM networks. The training of any neural network follows the same steps regardless of the type of neural network. Iteratively, the network is presented with the chosen input data, and computations as specified by the architecture of the neural network, the produced output which in this case is an estimate of blood pressure is compared against a simultaneously measured value of systolic or diastolic blood pressure to compute an error which is then used to update the weights or parameters of a neural network during training The ultimate objective is to iteratively improve the accuracy of values produced by the network by incrementally evolving the parameters within the neural architecture that is defined prior to initiation of training Recurrent neural networks are a type of neural networks where the output of one layer is recurrently presented as an input to the layer. These networks are specifically suitable for time series data inputs and predictions that need to be made using time series data as inputs. There are several nuanced variations to the basic architecture of these networks that may result in variants, but the basic idea of recurrence remains the same—present the output or part of the output of a layer as a part of the input to the layer so that there is memory in the network, or it remembers what value it saw before and modifies its predictions accordingly.

Finally, the output is obtained. The output of Method 2 is the same as the regression layer output as defined for Method 1. The output is an estimate of systolic, diastolic or both pressures associated with the same time stamp or time window as the presented input.

In Method 3 300 the input data 10 is processed through Extraction of waveform pattern characteristics in time and transform domains 310. The output of this step is Extracted Features 710 Next, feature selection occurs using the extracted features 710 combined with time synchronous target measurements from a sphygmomanometer or arterial line 740 which are input into the feature selection data when needed. The time synchronous target measurements are only included when the available set of inputs are inadequate to estimate blood pressures with errors less than ±6mm of Hg. The time synchronous measurements are reference blood pressure measurements that can be associated with the features that are extracted from the input data such that for each instance of a feature set extracted from the input data, there is an associated instance of blood pressure measurement available at the same time window or time stamp. An evaluation of which features are correlated to blood pressure is conducted during feature selection so that features that are strongly related to blood pressure and not to each other are chosen. If the features are related to each other, the model is more likely to overfit during training, which is the condition when the model is highly accurate for training data estimation of blood pressure, but inaccurate when applied to the testing data.

The strongly correlated features 730 are then selected from the output of 720. There are stablished feature selection algorithms such as minimum redundancy maximal relevance (MRMR), F-statistic or f-test, neighborhood component analysis, backward elimination, forward selection, random forests impurity-based importance, permutation feature importance, factor analysis, and relief algorithm for regression. It is possible to apply all of these different algorithms and then choose the best performing. In one preferred embodiment of the present invention, random forests impurity-based importance and MRMR are used to select features, with a preference for MRMR.

After the strongly correlated features 730 are selected, a model training and selection is conducted 750 using the output of the strongly correlated features 730 and simultaneously, the same Time synchronous target measurements from the sphygmomanometer or arterial line 740. The Time synchronous target measurements are used to compare with an instance of estimation of blood pressure by the model that is undergoing training against a ground truth value so that the model can be adjusted in a way that the error of estimation can be reduced incrementally. All training samples (each sample is an instance of features and associated ground truth measurement of blood pressure measurement) are presented iteratively.

Finally, the Output of the Process, in which the Systolic Predictor Model/Diastolic Predictor Model 760 is performed. In Method 3, features are patterns present within the time series waveform data—for example:—ECG has several characteristic waveforms such as R wave, S wave, P wave, T wave and so on. The extracted feature may be just the amplitude of the R wave because it is relevant to blood pressure. In contrast, S wave amplitude is not relevant. Therefore, it is possible to use only the extracted features with relevance to blood pressure and not the time-varying waveforms. Time-varying can be used directly without feature extraction for deep learning architectures also referred to as end-to-end machine learning architectures.

In Method 3 300, features present within the input data may be computed or extracted as predefined waveform patterns or time-varying quantities that are measured inputs that change over time that can be trended, such as but not limited to, distribution of amplitude and power in different frequency ranges of any of the input time-series data (with the time-series data referred to herein being part of the input data that is available for blood pressure estimation)These time-frequency analysis and computation methods 310 may involve any transformation methods such as Fourier, wavelet, short-time Fourier, cepstral analysis, empirical mode decomposition, or wavelet decomposition. In a preferred embodiment, wavelet decomposition is used for heart sound data and fourier transforms and filtering using infinite impulse response filters such as butterworth filters for ECG.

Time synchronous features obtained through the fusion of different types of input signals such as but not limited to ECG and heart sound are used to obtain features such as the R peak to S1, S2, S3, and S4 times, if present, which are reflective of pre-ejection and ejection times from the left ventricle of the heart during the isovolumic contraction period leading up to systole and diastole thereafter. In one preferred embodiment, the ECG heart sound feature specifically is a measure of time that has elapsed from the time of occurrence of the R wave peak of the ECG and the characteristic heart sounds. Time synchronous here means that such a time elapsed feature cannot be obtained unless both ECG and heart sound are recorded simultaneously or time synchronously because alignment of each heart beat's occurrence across ECG and heart sound is necessary. Otherwise, this feature cannot be extracted.

The input data are ECG and heart sounds. The ECG features are RR intervals and timing of the R peaks occurrence. The R peaks of the ECG waveform were detected algorithmically. The detected R peaks are used to determine the bounds of the RR intervals for each heartbeat. An ensemble average is computed for one minute of data centered on the measurement of the blood pressure from the reference device. Heart sound ensembles are defined based on the RR intervals extracted from the ECG waveform. The ensemble average was computed across one minute of data centered at the time of the blood pressure measurement from the reference device like the ECG waveform. The S1 and S2 times of occurrence and root mean square amplitudes are algorithmically extracted from the ensemble average. Based on the extracted timing of the R peak of the ECG and the S1 and S2 peaks of the heart sound, a timing feature is extracted.

These timing features are differently important for the prediction of systolic and diastolic pressures, and their respective importance may vary based on confounders such as arterial stiffness or pharmacological effects of vasoactive drug therapy. Levels of activity and absolute body posture with the inclination of the upper and lower body relative to the ground, as measured by an accelerometer, gyroscope, or an inertial measurement unit with the ability to measure movement in at least three axes inclusive of static measurement of the acceleration due to gravity. Altitude relative to sea level and geographic location impacts blood oxygenation and sympathetic responses such as vasodilation to compensate for any reduction of oxygen saturation in the blood due to lowering the concentration of oxygen at higher altitudes, or presence of endemic pollutants, gases, or particles that may affect blood pressure.

The preferred embodiment of the present invention is the personalized method of estimating blood pressure using Method 3 for initial model development and the personalization embodiment to fine tune the models performance for a an individual patient.

In Method 4 400, the input data is subjected to unsupervised clustering methods leading to clustering of data in different levels of granularity of time 410. The output of 400A starts with input data 10 with the input data subjected to unsupervised clustering methods leading to clustering of data in different levels of granularity of time 410 which is then combined in the Extracted Features step 710. Next, feature selection occurs using the extracted features 710 combined with Time synchronous target measurements from a sphygmomanometer or arterial line 740 which are input into the feature selection data. The feature selection is then performed. Then strongly correlated features 730 are selected from the output of 720 after which a model training and selection is conducted 750 using the output of the strongly correlated features 730 and simultaneously, the same Time synchronous target measurements from the sphygmomanometer or arterial line 740. Finally, the Output of the Process, in which the Systolic Predictor Model/Diastolic Predictor Model 760 is performed.

In the Method 4, all input data may be subjected to unsupervised learning methods to form clusters such as those based on neural networks such as autoencoders, self-organizing maps or adaptive resonance theory-based neural networks, or machine learning techniques such as k means clustering, Gaussian mixture models, naïve Bayes, density-based or model-based techniques.

With a set of features extracted, if it is known based on the cluster classification (which will be the result of the listed methods applied directly to the set of features extracted from the training data—at the end of applying the method, a transformation is obtained that will output the cluster membership for an input of any instance of features extracted and presented to the transformation) that certain clusters of features should lead to estimating certain ranges of blood pressure , then the determination through unsupervised learning that a particular instance of features extracted should mean that the blood pressure ought to be in a certain range can guide the training of models to use the cluster assignment information to estimate blood pressure. Essentially, in this case unsupervised learning is used to find patterns within the features that are extracted that can be used to estimate blood pressure.

The clusters are defined in the input feature space, which is the mathematical definition of space. For example, if 2 features are extracted in total, then the feature space is 2-dimensional. It is defined as the full range and permutation of all possible values that can be taken by any instance of the 2 features like a flat 2-D grid of infinite expanse. The cluster classifications, in turn, are then used for regression-based blood pressure prediction models as features.

From here, Method 4 follows the same path as the previous Methods. Method 4 can continue with further feature selection, training and selection with an additional embodiment including the option to personalize models using data from an individual to tune models.

In Method 5 500, one dimensional time series to 2 or higher dimensional encoding or sequential mapping 210 is performed on the input data 10 and then the output data of step 210 is applied to a subset of pre-trained neural network layers on a different dataset i.e., a dataset that is different from the data available for blood pressure estimation. These neural networks are trained to perform tasks such as image classification on a dataset consisting of 1000s or more images and their associated labels such as cats, dogs, streetlights and so on, and may use deep neural network architectures consisting of several layers of neural networks. The output of the penultimate or earlier layer is used as the extracted features 510. The choice of layer is made through exhaustive searching through the layers to find a stage or combinations of stages where the output of the pre-trained neural network is useful as a feature for the models trained later during feature selection step. The output of step 510 are the extracted features 710. Next, feature selection occurs using the extracted features 710 combined with ime synchronous target measurements from a sphygmomanometer or arterial line 740 which are input into the feature selection data. The feature selection is then performed. Then, as explained in detail in Method 3 above, strongly correlated features 730 are selected from the output of 720 after which a model training and selection is conducted 750 using the output of the strongly correlated features 730 and simultaneously, the same Time synchronous target measurements from the sphygmomanometer or arterial line 740. Finally, the Output of the Process, in which the Systolic Predictor Model/Diastolic Predictor Model 760 is performed.

In Method 5 500, a subset or all layers of a trained neural network 510 may be used as a feature extractor. An example embodiment of this approach may involve the transformation of the input layer of a pre-trained network to accept the input from this device and a subset of all the remaining layers of such a pre-trained neural network or all layers, but the output layer may be retained in the model. The output of the penultimate layer of this neural network may be used as a transformation to extract features that may be the input of a subsequent regression model to predict systolic and diastolic blood pressures. In this embodiment, the pre-trained neural network implements a transfer function for the extraction of features. Examples of such neural networks are convolution neural networks, recurrent neural networks, and encoder networks. The output of 510 is a set of features which are used as extracted features 710 which are then subjected to feature selection and then model training.

In Method 6 600, the input data 10 is applied to the discriminator of a trained generative network 610 to obtain the extracted features 710 Next, feature selection occurs using the extracted features 710 combined with Time synchronous target measurements from a sphygmomanometer or arterial line 740 which are input into the feature selection data. The feature selection is then performed. Then strongly correlated features 730 are selected from the output of 720 after which a model training and selection is conducted 750 using the output of the strongly correlated features 730 and simultaneously, the same Time synchronous target measurements from the sphygmomanometer or arterial line 740. Finally, the Output of the Process, in which the Systolic Predictor Model/Diastolic Predictor Model 760 is performed.

The sixth method includes training a generative neural network that consists of structures involving a generator component and a discriminator component, such as a Generative Adversarial Network (GAN). The generator component is a neural network that takes as input a random input signal that can be generated using a pseudo random generator with a white noise distribution and generates the input data that is part of the training data set. This component generates the input data and is trained to generate the input data with minimal error. The discriminator component is also a neural network that takes as input the output of a generator component and determines whether the output is acceptably close to the input data using a cost function that evaluates the amount of error.

Given a training set, this technique learns to generate new data with the same statistics as the training set. It is possible, for instance, to use a GAN and its discriminator output to extract features. For example, a GAN trained on photographs can generate new photographs that look at least superficially authentic to human observers, having many realistic characteristics. The core idea of a GAN is based on the “indirect” training through the discriminator, another neural network that can tell how “realistic” the input seems, which itself is also being updated dynamically. This means that the generator is not trained to minimize the distance to a specific image, but rather to fool the discriminator, hence adversarial.

The input data applied to the discriminator generates a set of features that can be used to train another neural network or machine learning model to predict blood pressure. The output of the discriminator component is a set of features and can be 710. The remainder of the process of Method is the same as other methods discussed above.

Similarly, the methods of the present invention for personalizing or continuously improving upon the blood pressure predictions include, but are not limited to, personalized blood pressure models through one of the following approaches:

Through transfer learning, wherein a pre-trained model on a population may be trained further with data from an individual to generate a blood pressure prediction model that is uniquely tailored for that individual. In preferred embodiments, the pretrained model is a large population having at least 50 subjects. In more preferred models, the large population has at least 85 subjects. In preferred embodiments, approximately 25% of the population has normal blood pressure, (systolic below 120 mm of Hg and diastolic below 80 mm of Hg), approximately 25% of the population has pre-hypertensive blood pressure values (systolic between 120 and 140 mm of Hg or diastolic between 80 mm of Hg and 90 mm of Hg), approximately 25% of the population has stage 1 hypertension blood pressures (systolic between 140 and 160 mm of Hg, and diastolic between 90 mm of Hg and 100 mm of Hg), and approximately 25% of the population has stage 2 hypertension (systolic above 160 mm of Hg and diastolic above 100 mm of Hg). For example, in a population of 85 patients. At least 21 subjects have normal blood pressure, 21 subjects have pre-hypertensive blood pressure values, 21 subjects have stage 1 hypertension blood pressures (systolic between 140 and 160 mm of Hg, and diastolic between 90 mm of Hg and 100 mm of Hg, and 21 subjects have stage 2 hypertension. The model is pretrained through one of the methods described above. A model trained to estimate blood pressure using Methods 1, 5, or 6 can be trained starting from the trained state with training data that is collected from a single patient or subject. This additional training is referred to as transfer learning where the learning that the model accumulates from the training data consisting of several patients is transferred and its accuracy is further improved so that it is specifically accurate for an individual. This can be described as a method to personalize and calibrate the blood pressure estimation models to work particularly well for these subjects. In a preferred embodiment, the model is trained using Method 3.

Additional input data may be obtained through a continuous blood pressure measurement technique for a few minutes to a few days, such as an arterial line and the blood pressures measured may be used as a personalized training set for an individual and an exploration of all aforementioned feature extraction and model training methods may be applied to create a customized blood pressure prediction. The additional input data is added to feature selection and to model training and selection for personalization of blood pressure models.

Model improvements, conditioning, corrections to consider any confounders arising because of patients' health condition including cardiac, pulmonary, respiratory, circulatory, acoustic, hemodynamic, movements, and blood flow characteristics and metrics including, but not limited, to the features mentioned in Table 2. Table 2 is a more exhaustive list of features than the preferred features of Table 1, and describes e.g. all features that can derived from SimpleSense™ device captured data. During the feature selection step, features are evaluated based on their relevance to the estimated output (systolic and diastolic blood pressure). A determination of a preferred number of features to use can be assessed through experiments with different sets of features and observing the trained model performance in terms of the accuracy of the estimated blood pressures compared to the reference measurements that were taken while the input data was being simultaneously captured. This is an exhaustive search to evaluate every permutation and combination of features as inputs to models. In other words, the minimum or preferred number of input data to be used can be determined by how many inputs are needed to achieve an accuracy or an error of estimation of blood pressure below about 6 mm of Hg, with the error measured as the mean absolute difference, or with the mean error below about +5 mm of Hg and standard deviation of error is less than about +8 mm of Hg.

TABLE 2
Exemplative Features obtained from SimpleSense™ device

| Name of Feature | Source | Description |
|---|---|---|
| Atrial Electrical Activity | ECG | Duration and amplitude |
| | | of P wave of ECG |
| | | waveform |
| Ventricular Electrical Activity | ECG | Duration and amplitude |
| | | of QRS wave of ECG |
| | | waveform |
| PR interval or Atrio- | ECG | Time elapsed between P |
| ventricular conduction interval | | wave and R wave |
| | | occurrence in an ECG |
| QRS measures | ECG | Amplitude, duration and |
| | | axis of QRS waves of |
| | | ECG. |
| ST-T wave measures | ECG | ST segment amplitude, |
| | | duration and slope in |
| | | ECG waveform |
| S1, S2, S3, S4 sounds | Heart sound | Prescence, amplitude, |
| | | magnitude, loudness, |
| | | time durations of heart |
| | | sounds |
| Other heart sounds and noise | Heart sound | Prescence, amplitude, |
| | | magnitude, loudness, |
| | | time durations of heart |
| | | sounds and noise |
| Patient Activity Score | Actigraphy | Measure of physical |
| | | activity performed by |
| | | patient like walking, |
| | | climbing stairs or more |
| | | intense exercise |
| Posture | Actigraphy | Measure of the absolute |
| | | posture maintained |
| Cardiac Output | ICG | Measure of the volume |
| | | of blood pumped from |
| | | the heart in a minute. It is |
| | | the product of the |
| | | volume of blood pumped |
| | | out be the left ventricle |
| | | of the heart and the heart |
| | | rate. |
| Stroke Volume | ICG | Measure of volume of |
| | | heart pumped from the |
| | | ventricle of the heart. |
| Cardio-vascular Pressures | ICG | Measure of the |
| | | maximum pressure in the |
| | | blood vessels following a |
| | | heart muscle contraction |
| | | that causes blood flow |
| | | and pressure between |
| | | consecutive contractions. |
| Patient Geographic Location | Smart devices | Global Positioning |
| and Altitude | | Satellite (GPS) location |
| | | of the patient from the |
| | | patient’s smart device. |
| Pulmonary Measures | ICG | Pulmonary measures |
| | | include tidal volume - |
| | | volume of air inhaled or |
| | | exhaled during normal |
| | | breathing, and rate of |
| | | breathing, whether the |
| | | patient is experiencing |
| | | shortness of breath |
| | | wherein the respiratory |
| | | rate is high, but the |
| | | volume of air displaced |
| | | from the lungs is low. |
| Minute Ventilation | ICG | Measure of the amount |
| | | of air displaced by the |
| | | lungs in a minute. |
| Shortness of Breath | ICG and ECG | Measure of the amount |
| | | of air inhaled or exhaled |
| | | and rate of respiration. |
| Exercise Tolerance | ECG and ICG | Measures the changes in |
| | | heart rate and respiration |
| | | while performing |
| | | activities like a 6-minute |
| | | walk |
| Heart Rate | ECG | Measures the total |
| | | number of heart beats per |
| | | minute |
| Heart Rhythm including P, Q, | ECG | Measures the regularity |
| R, S, T analysis | | of the heartbeat. |
| Transthoracic impedance | ICG | Measures the electrical |
| | | impedance of the thorax |
| | | or chest of the patient. |
| Skin Conductance | GSR | Measure the conductance |
| | | or resistance of the skin |
| Blood oxygen levels | PPG | Volumetric changes in |
| | | arterial blood which is |
| | | associated with cardiac |
| | | activity, variations in |
| | | venous blood volume |
| Body or skin temperature | Temperature | Human body or skin |
| | | temperature |

A plurality of model inputs could include, but not be limited to, height of the patient, a weight of the patient, a gender of the patient, an age of the patient, a medical history and physical examination records of the patient, a medical status of the patient, a body mass index (BMI) of the patient, an ethnicity of the patient, a medical prescription history of the patient, a medical prescription status of the patient, types of BP treatments and medications received by the patient, types of medical treatments for health issues and insurance or claims information previously received by the patient, diet information for the patient, psychological history of the patient, and a genetic indicator of the patient, biomarkers of the patient along with other EMR information.

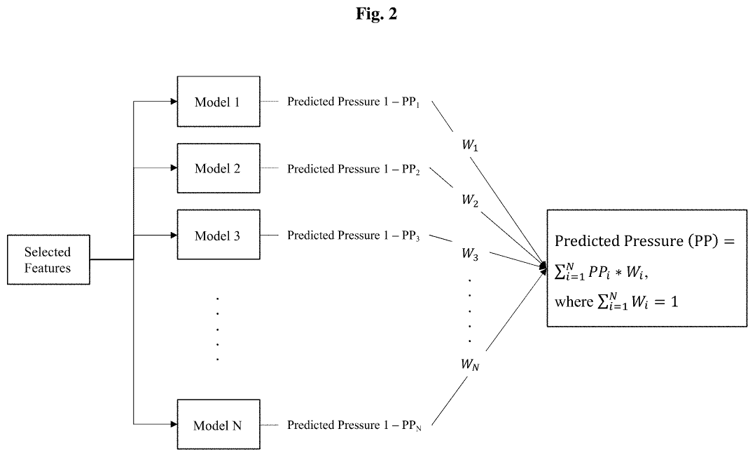

FIG. 2 shows exemplary implementation of an ensemble model that combines outputs from multiple models using a weighted sum with the same features selected in Methods 1-6 of FIG. 1, which are discussed in detail above. In FIG. 2, each model has a different architecture and hyperparameter choice for estimation of pressure, so each model produces its own estimate of pressure for systolic or diastolic referred to as PP1, PP2, etc. W1, W2, and W3 are all weights that are proportional to the importance defined by the accuracy that was achievable by each of the models that product predicted pressures PP1, PP2 etc. The predicted pressure formula illustrates that a weighted sum of outputs from several models referred to as ensemble regression can also be used to estimate blood pressures. The formula mathematically defines the weighted summation operation. N in the formula can be a number from 1 to 10 for practical purposes. Additional models beyond 10 are not likely to improve overall performance If more than 10 models are needed then ensembling is not the best method to use and alternate methods involving deep learning architectures should be explored.

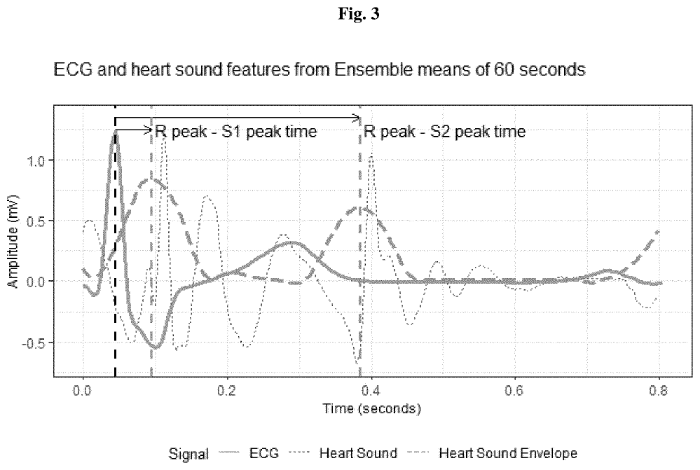

FIG. 3 shows exemplary multi-modal time synchronous features extracted from actual input data. The time elapsed from R peak of ECG to S1 sound of heart sounds, and from R peak of ECG to S2 sound of heart sound are indicative of electromechanical coupling in single heartbeat associated with diastolic, and systolic phases of hemodynamics within the heart, respectively.

FIG. 4 shows an actual feature selection process for a systolic blood pressure prediction model using impurity-based feature importance using a random forest regression model. The impurity-based feature importance ranks all the numerical features to find the most important features that are needed for blood pressure estimation. As a means of selecting the most important features, preferably, features that had more than 2% overall relative numeric importance can be selected. The impurity-based feature importance ranks all the numerical features to find the most important features that are needed for blood pressure estimation. The feature importance values are computed as the mean and standard deviation of accumulation of the impurity decrease within each tree. The impurity-based feature importance algorithm counts the times a feature is used to split a node while training a random forest regression model, weighted by the number of samples it splits. Essentially, the earlier a feature is deemed important for a split or a decision, the more important that feature is to estimating the blood pressure.

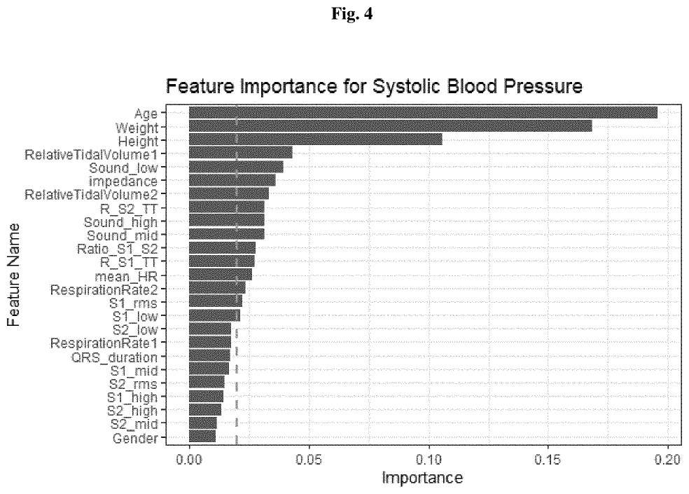

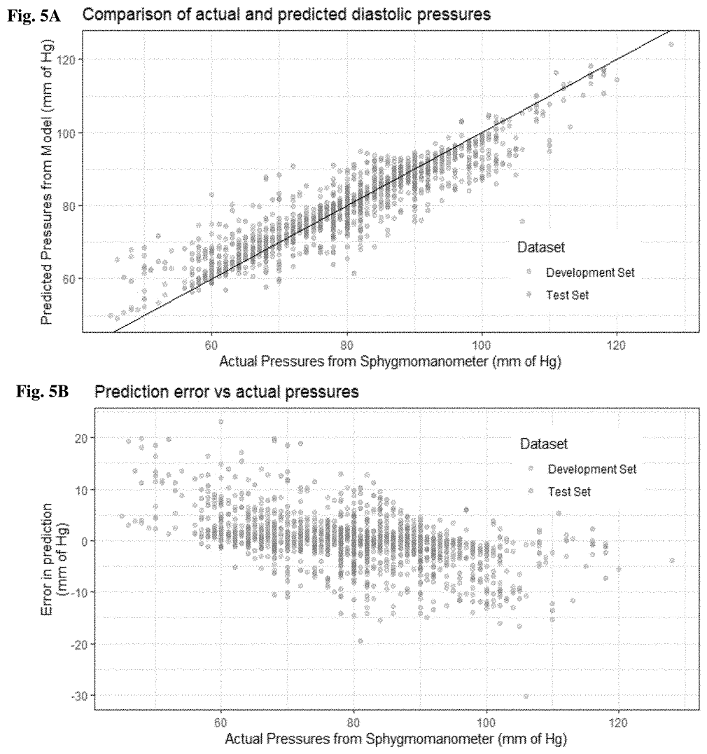

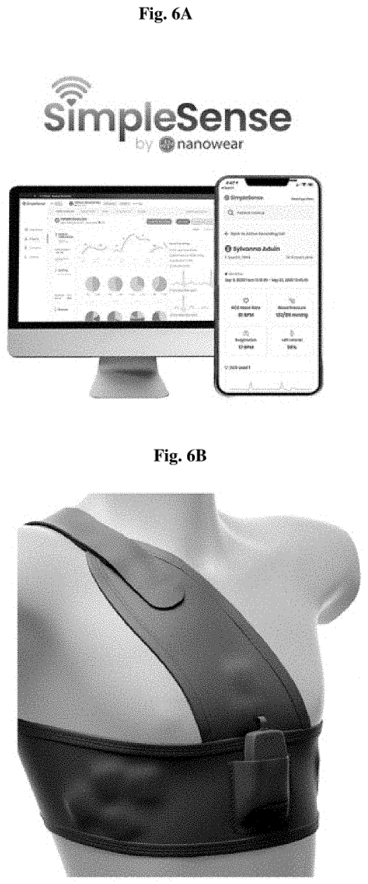

FIGS. 5A and 5B show an evaluation of performance of a trained diastolic pressure prediction model, by showing a comparison of actual and predicted measures of systolic blood pressures from an exemplary blood pressure prediction model. FIG. 5A is a scatter plot of predicted vs actual SBP for both training and test set. The training set in this case is the input data used to develop the model and test set is the input data set used to evaluate the performance so that the training and test set are independent of each other, i.e., obtained at different times from different patients. The predicted pressures are closely in alignment with the actual target measurements of blood pressure as seen in FIG. 5A. Blood pressure values were available from a sphygmomanometer at specific instances of time by trained nurses. Simultaneously, data from a SimpleSense™ device was collected. The exemplary implementation uses the preferred method to determine a model which is used to predict blood pressure values using the SimpleSense™ device and the demographics data available at the stated instances of time when the nurses took readings from a sphygmomanometer. Prediction error is the difference between the predicted blood pressure and the actual blood pressure from a sphygmomanometer measurement. FIG. 5B is a scatter plot plotted as actual pressures as measured by the sphygmomanometer versus the prediction error.

Example Using SimpleSense™ Device and Software Platform and process of Method 3

Targeted maintenance of blood pressure for hypertensive patients requires accurate monitoring of blood pressure at home. Use of multi.parametric vital signs ECG, heart sounds, and thoracic impedance for blood pressure estimation at home has not been reported previously. Two ensemble regression tree models trained using data from 120 subjects was used to estimate systolic blood pressure and diastolic blood pressure as described below.

In an observational multi-site study. 120 subjects (female (N=61.52%)) between 18 and 83 years of age were recruited with the following stratification (Normal (20%), prehypertensive (37%), stage 1(26%), am stage 2 (18%). From these subjects, 1686 measurements of blood pressure from a sphygmomanometer were associated with simultaneously acquired signals from the SimpleSense™ device. An ensemble of tree-based models was trained with inputs as metrics derived from the muitiparametric and patient demographics data. A test Mean Absolute Difference (MAD) of ±6.38 mm of Hg and ±5.10 mm of 14g were obtained for systolic and diastolic blood pressures (systolic blood pressure; diastolic blood pressure), respectively. Comparatively, the MAD for wrist-worn blood pressure cuff OMRON BP6350 (GUDID—10073796266353) was ±8.92 mm of Hg and ±6.86 mm of Hg, respectively, Machine learning models trained to use lultiparametric data can monitor systolic blood pressure and diastolic blood pressure without the need for calibration, and with accuracy levels comparable to at-home cuff-based blood pressure monitors.

### Study Design

A prospective multicenter ton-randomized observational study was performed and consisted of executing a protocol with each subject involving activities that modulate the blood pressure for a duration of up to 1 hour and 30 minutes. The study protocol was approved by the independent Institutional Review Board (IRB) Advatya Inc. (Columbia, Md.). The study was conducted according to the stipulations of the 1964 Declaration of Helsinki and in accordance with relevant FDA regulations. Written informed consent was sought and obtained from all subjects and all subjects completed the study. The accuracy of the estimated blood pressure was evaluated following a validation plan based on the IEEE standard 1708a-2019. (IEEE Standard for Wearable, Cuffless Blood Pressure Measuring Devices—Amendment 1, IEEE Std 1708a-2019 (Amendment to IEEE Std 1708-2014), 1-35 (2019)).

### Patient Population

From Feb. 8, 2021 to Jul. 16, 2021, subjects were screened for eligibility and recruited. The targeted subject selection and stratification are presented in Table 3. The subjects were sorted into four cohorts—normal, prehypertensive, Stage 1 hypertension, and Stage 2 hypertension-based on the specified range of blood pressure in Table 3. The endpoint of the blood pressure classification was determined according to the. JNC 7 report. (Chobani an, A. V. et al., The seventh report of the Joint National Committee on Prevention, Deter Evaluation, and Treatment of High Blood Pressure: The INC 7 report, JAMA, 289,2560-2572, (2003)). The blood pressure used for classification was the entry blood pressure measured at the beginning of the test. Three measurements were taken from the subject sitting with the elbow, back of the elbow, and back resting on a chair. The averaged value was used as the entry blood pressure to determine the subject's blood pressure classification.

TABLE 3
Subject selection requirements

| | Systolic blood | | Diastolic blood | Subjects | Subjects |
|---|---|---|---|---|---|
| Blood pressure | pressure | | pressure | in | in the |
| classification | (mmHg) | | (mmHg) | Dev. set | test set |
| Normal | <120 | and | <80 | 5 | ≥16 |
| Prehypertension | 120-139 | or | 80-89 | 5 | ≥16 |
| Stage 1 | 140-160 | or | 90-100 | 5 | ≥16 |
| hypertension | | | | | |
| Stage 2 | ≥160 | or | ≥100 | 5 | ≥16 |
| hypertension | | | | | |

Gender: At least 26 males and 26 females

### Study Procedure

Two trained observers performed all measurements sing the SimpleSense™ device and gold standard sphygmomanometer for each subject. The training of the observers was as described in Section 5.2.2 of ISO 81060-2019.

### Blood Pressure Measurement

First, the subjects wore the SimpleSense™ system. The device recorded data while the two observers took the blood pressure measurements using a standard sphygrnomanorneter and Omron blood pressure wrist cuff GUDID 10073796266353. The simultaneously and synchronously acquired data from SimpieSense™ was used to calibrate the algorithm against the blood pressure measurements from the sphygmomanometer.

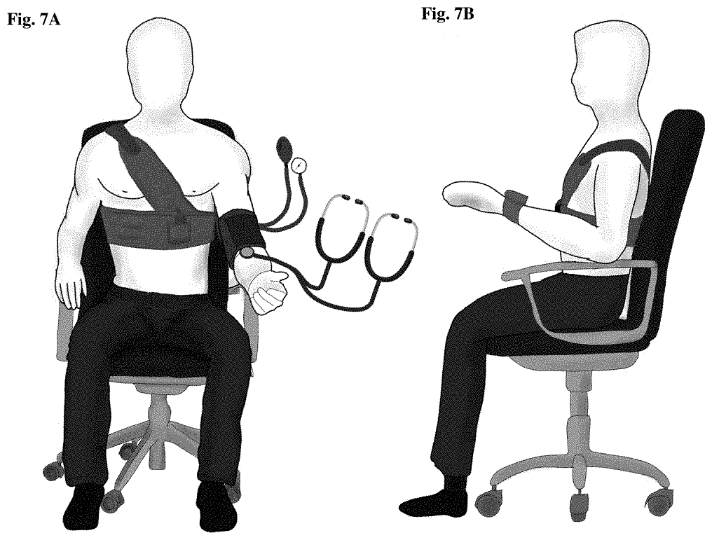

Reference measurements were provided by the k trained observers and measured simultaneously with one reference sphygmomanometer (using a “Y” connector) as shown in FIG. 7A. The sphygmomanometer used as the reference standard met the requirement in ISO 81060-2:2019 or ANSI/AAMI SP10. Systolic blood pressure and diastolic blood pressure measurements with the sphygmornanometer were determined using the Phase 1 and Phase 5 Korotkoff sounds, respectively. The observers set up the sphygmomanometer and Omron wrist cuff, as shown in FIG. 7B. The wrist cuff and the sphygmomanometer were worn on the same (left) arm. All measurements were recorded to the nearest two mmHg. If both measurements from the two observers are no more than four mmHg apart, the mean value of the two was used as the reference measurement. Otherwise, the measurement was retaken. If either observer detected significantly irregular heart rhythm, that reading was excluded. The time of blood pressure measurement was recorded accurately to the nearest minute. The supplementary material includes the detailed test procedure.

Simultaneous measurement was used for the sphygmomanometer and SimpleSense™ device. The left arm was used for reference measurement. By design, the SimpleSense™ system is a cuffless device and does not rely on an arm or wrist cuff for blood pressure measurement. It can record all the physiological data during the inflation and deflation of the reference sphygmomanometer without any mutual interference. At least a 60-second delay was observed between consecutive readings from the sphygmomanometer to avoid venous congestion.

Sequential measurement was used for the Omron Wrist Cuff. When the wrist cuff device is used with the sphygmomanometer reference measurement, either arm will interfere with the reference device measurement. The Omron wrist cuff measurement was done at least 60 seconds after the reference measurement but not more than 90 seconds to avoid increased variability due to expected physiological trends.

### Modulation of Blood Pressure

Subjects were asked to perform activities that modulated blood pressure to increase the dynamic range of observed systolic blood pressure and diastolic blood pressure. The lowering of blood pressure was induced by asking the subject to sit with feet raised on a stool or chair and hold a warm water bottle (warm stimulus) in their hand wrapped with an insulating cloth. The increases in blood pressure were induced by asking the subject to walk briskly (mild exercise), as physically able, for about 10 min and holding an ice pack (cold presser) their hands for 5-10 min as tolerated. Three consecutive recordings of simultaneous and sequential measurements of blood pressure were performed after each of these blood pressure modulating activities.

### Data Preparation and Processing

The SimpleSense™ data for each patient was first subjected to a data quality assessment. Segments of data that were of insufficient quality due to the presence of noise due to any movements were removed from further consideration. From the recorded data that was deemed to be of acceptable quality, within a 300-second window preceding a time-stamped observation of systolic blood pressure and diastolic blood pressure by the observers using the gold standard sphygmomanometer, a 60-second window of data with acceptable quality was extracted and associated with that observation. These steps were performed for each recorded blood pressure value from the gold standard device. Thus, a dataset was prepared with the 60-second-long; segments of SimpleSense™ device data and the associated target systolic blood pressure and diastolic blood pressure values for training the SimpleSense™-BP algorithm. The systolic and diastolic reference measurements acquired from the gold standard device were then randomly split into two sequestered sets of 80%/20% (training/test).

Mean Absolute Difference (MAD) (Eq. 1), Mean Absolute Percentage Difference (MAPD) (Eq. 2), and Root Mean Square Error (RMSE) (Eq. 3) are used to analyze the performance of the models. The statistical aspect of the criteria is discussed in IEEE 1708-2019a.

$$MAD = ( {\sum }_{i = 1}^{n} \vert {p}_{i} - {y}_{i} \vert ) / n \qquad ( 1 )$$

$$MAPD = ( {\sum }_{i = 1}^{n} 100 * \vert {p}_{i} - {y}_{i} \vert / {y}_{i} ) / n \qquad ( 2 )$$

$$RMSE = \sqrt{\frac{{\sum }_{i = 1}^{n} {( {p}_{i} - {y}_{i} )}^{2}}{n}} \qquad ( 3 )$$

where pᵢpᵢ is the test device measurement, yᵢyᵢ is the average of the adjacent two reference measurements taken before and after device measurement as defined in ISO 81060-2:2019, and n is the data size. Applying a constant accuracy limit to measurements at both the low and high ends of blood pressure is biased because the variability nay increase at both extremes of blood pressure. An incorrectly significant error may be introduced. Therefore, MAPD is used in addition to MAD.

Further, the contribution of the SimpleSense™ device was evaluated through measured inputs and demographics for blood pressure estimation in comparison to models that use only demographics data. The comparison method follows the recommendation by Mukkamala et al. (Mukkamala, R. et al., Evaluation of the accuracy of cuffless blood pressure measurement devices: Challenges and proposals, Hypertension 78, 1161-1167, (2021)). The description of the method to compare models described by Natarajan et al. presents a convenient method to measure overall accuracy through bootstrap RMSE. First, we trained a reference model that uses only the demographics data (Age, Gender, Height, and Weight) on the same training data as SimpleSense™-BP to estimate systolic blood pressure and diastolic blood pressure. (Photoplethysmography fast upstroke time intervals can be useful feature cuff-less measurement of blood pressure changes in humans, IEEE Trans. Biomed. Eng., 69, 53-62, (2022), The reference model training procedure used the same training hyperparameters for the automl framework as the SimpleSense™-BP model. 10,000 random samples of the test set measurements with replacement were taken. The number of samples per iteration was equal to the size of the test set. For each sample, the Root Mean Square Error (RMSE) was computed for each model and the difference between the RMSEs of the two models for comparison as (SimpleSense™-BP estimated blood pressure—Demographics only estimated blood pressure for systolic blood pressure and diastolic blood pressure). The criteria to support the superiority of one model. over the other was that the difference in RMSE errors should have an upper 95% Confidence Interval (CI) less than 0 mmHg.

### SimpleSense™ Data Analysis and Feature ExplorationECG Features

The ECG features were RR intervals and timing of the R peaks occurrence. The R peaks of the ECG waveform were detected algorithmically. The detected R peaks were used to determine the bounds of the RR intervals for each heartbeat An ensemble average was computed for one minute of data centered on the measurement of the blood pressure from the reference device.

### H Sound Features

Heart sound ensembles were defined based on the RR intervals extracted from the ECG waveform. The ensemble average was computed across one minute of data centered at the blood pressure measurement from the reference device like the ECG waveform. The S1 and S2 times of occurrence and root mean square amplitudes were algorithmically extracted from the ensemble average.

### ECG and Heart Sound Features

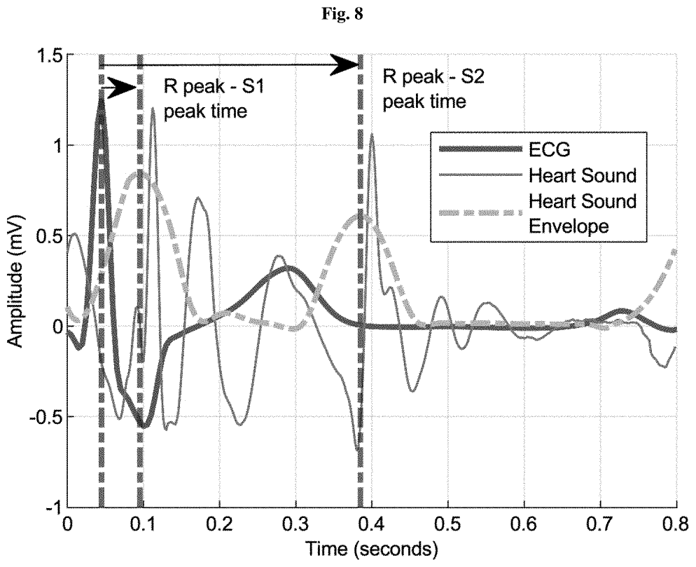

Based on the extracted timing of the R peak of the ECG and the S1 and S2 peaks of the heart sound, a timing feature was extracted. The time elapsed between the R peak and the 51 and S2 sounds is illustrated in FIG. 8. FIG. 8 further summarizes the Inetrics as derived from ECG and heart sound.

Table 4 provides a list of features, their source, and the descriptions of the features. The SimpleSense™ device acquires two channels of thoracic impedance, with one channel spanning the thorax from the right shoulder to the lower-left abdomen and the other around the abdomen. Respiration-related features are obtained for each channel and are treated as independent features because of the type of respiration effort measured by the two channels—thoracic and abdominal.

TABLE 4
List of features, descriptions, and evidence correlating feature and SBP and DBP in the
literature

| | | | | Studies in the literature that have |
|---|---|---|---|---|
| | | | | correlated features to either SBP or |
| # | Name | Source | Description | DBP, or both |
| 1 | Mean | Impedance | Mean respiration rate | Respiration is known to influence BP¹⁻³. |
| | respiration | | computed over a minute of | Slow breathing may result in potential |
| | rate 1 | | thoracic impedance data | improvement for patients with essential |
| | | | from channel 1 | hypertension⁴. |
| 2 | Mean | | Mean respiration rate | |
| | respiration | | computed over a minute of | |
| | rate 2 | | thoracic impedance data | |
| | | | from channel 2 | |
| 3 | Relative | | Mean of the range of | |
| | Tidal | | thoracic impedance values | |
| | Volume 1 | | measured within a minute of | |
| | | | SimpleSense data from | |
| | | | channel 1. These ranges are | |
| | | | computed over 30-second | |
| | | | segments with 50% overlap. | |
| 4 | Relative | | Mean of the range of | |
| | Tidal | | thoracic impedance values | |
| | Volume 2 | | measured within a minute of | |
| | | | SimpleSense data from | |
| | | | channel 2. These ranges are | |
| | | | computed over 30-second | |
| | | | segments with 50% overlap. | |
| 5 | Mean | | Mean of the thoracic | Thoracic impedance is a non-invasive |
| | thoracic | | impedance values from | method of assessing fluid status and |
| | impedance 1 | | channel one over one | hemodynamics. The potential utility of |
| | | | minute. | impedance and its first derivative |
| | | | | impedance cardiography in the |
| | | | | hemodynamic assessments of patients |
| | | | | with heart failure is prevalent in the |
| | | | | literature⁵. |
| 6 | R to S1 time | Heart sound | Mean of the time intervals | These timing parameters are scarcely |
| | | and ECG | between the occurrence of | explored in literature due to the scarcity of |
| | | | the R peak in the ECG and | simultaneous measurement of heart |
| | | | the S1 heart sound peak in | sounds and ECG. From a physiological |
| | | | the Heart sound signal over a | standpoint, R-S1 and R-S2 timings are |
| | | | minute of data. | reflective of the systolic and diastolic |
| 7 | R to S2 time | | Mean of the time intervals | timings encompassing the pre-ejection |
| | | | between the occurrence of | time. |
| | | | the R peak in the ECG and | |
| | | | the S2 heart sound peak in | |
| | | | the Heart sound signal over a | |
| | | | minute of data. | |
| 8 | Mean | Heart sound | Average interval in seconds | Heart rate and heart rate variability are |
| | interbeat | | between heartbeats. Both | known modulators of blood pressure, and |
| | interval | | ECG and Heart sounds are | their non-linear coupling relationship is |
| | | | used to compute the mean | yet to be established^{6,7}. |
| | | | heart rate. | |
| 9 | The ratio of | | The ratio of S1 RMS and S2 | A novel feature explored in this study. |
| | S1 RMS to | | RMS | |
| | S2 RMS | | | |
| 10 | S1 RMS | | Mean of the root mean | There are several studies in the literature |
| | | | square (RMS) amplitude | that have sought to associate characteristic |
| | | | over a 125-millisecond | heart sound amplitudes with SBP and |
| | | | window centered at the time | DBP. The list of features explored in this |
| | | | of occurrence of the S1 | paper is an aggregation of all the reported |
| | | | sound of the heart sound. A | features in the literature^{8,9}. |
| | | | single value, the mean of the | |
| | | | RMS, is computed from a | |
| | | | minute of heart sound signal. | |
| 11 | S2 RMS | | Mean of the root mean | |
| | | | square (RMS) amplitude | |
| | | | over a 125-millisecond | |
| | | | window centered at the time | |
| | | | of occurrence of the S2 | |
| | | | sound of the heart sound. A | |
| | | | single value, the mean of the | |
| | | | RMS, is computed from a | |
| | | | minute of heart sound signal. | |
| 12 | S low | | Sum of amplitudes in the | |
| | | | frequencies from 50 to 100 | |
| | | | Hz relative to total amplitude | |
| | | | across all frequencies | |
| 13 | S mid | | Sum of amplitudes in the | |
| | | | frequencies from 125 to 175 | |
| | | | Hz relative to total amplitude | |
| | | | across all frequencies | |
| 14 | S high | | Sum of amplitudes in the | |
| | | | frequencies from 175 to 250 | |
| | | | Hz relative to total amplitude | |
| | | | across all frequencies | |
| 15 | S1 low | | Sum of amplitudes in the | |
| | | | frequencies from 50 to 100 | |
| | | | Hz relative to total amplitude | |
| | | | across all frequencies for a | |
| | | | sequence of 125 | |
| | | | milliseconds centered on the | |
| | | | S1 peak sound | |
| 16 | S1 mid | | Sum of amplitudes in the | |
| | | | frequencies from 125 to 175 | |
| | | | Hz relative to total amplitude | |
| | | | across all frequencies for a | |
| | | | sequence of 125 | |
| | | | milliseconds centered on the | |
| | | | S1 peak sound | |
| 17 | S1 high | | Sum of amplitudes in the | |
| | | | frequencies from 175 to 250 | |
| | | | Hz relative to total amplitude | |
| | | | across all frequencies for a | |
| | | | sequence of 125 | |
| | | | milliseconds centered on the | |
| | | | S1 peak sound | |
| 18 | S2 low | | Sum of amplitudes in the | |
| | | | frequencies from 50 to 100 | |
| | | | Hz relative to total amplitude | |
| | | | across all frequencies for a | |
| | | | sequence of 125 | |
| | | | milliseconds centered on the | |
| | | | S2 peak sound | |
| 19 | S2 mid | | Sum of amplitudes in the | |
| | | | frequencies from 125 to 175 | |
| | | | Hz relative to total amplitude | |
| | | | across all frequencies for a | |
| | | | sequence of 125 | |
| | | | milliseconds centered on the | |
| | | | S2 peak sound | |
| 20 | S2 high | | Sum of amplitudes in the | |
| | | | frequencies from 175 to 250 | |
| | | | Hz relative to total amplitude | |
| | | | across all frequencies for a | |
| | | | sequence of 125 | |
| | | | milliseconds centered on the | |
| | | | S2 peak sound | |
| 21 | QRS | ECG | Number of samples of the | Mixed evidence is reported on the |
| | duration | | ECG data between the first | association between QRS duration and |
| | | | zero crossing before QRS | blood pressure¹⁰. In this paper, empirical |
| | | | complex and first zero | evaluation of the utility of QRS duration |
| | | | crossing after the QRS | was explored. QRS duration was found to |
| | | | complex - denotes the | be unimportant as a predictor for both |
| | | | demarcation of the QRS | SBP and DBP. |
| | | | complex | |
| 22 | Age | Demographics | Age of the subject | The correlation between age, gender, |
| 23 | Height | Demographics | Height of the subject | BMI, and BP has been established in |
| 24 | Weight | Demographics | Weight of the subject | several studies¹¹⁻¹³. |
| 25 | Gender | Demographics | Gender of the subject | |

### Results

### Subject Characteristics

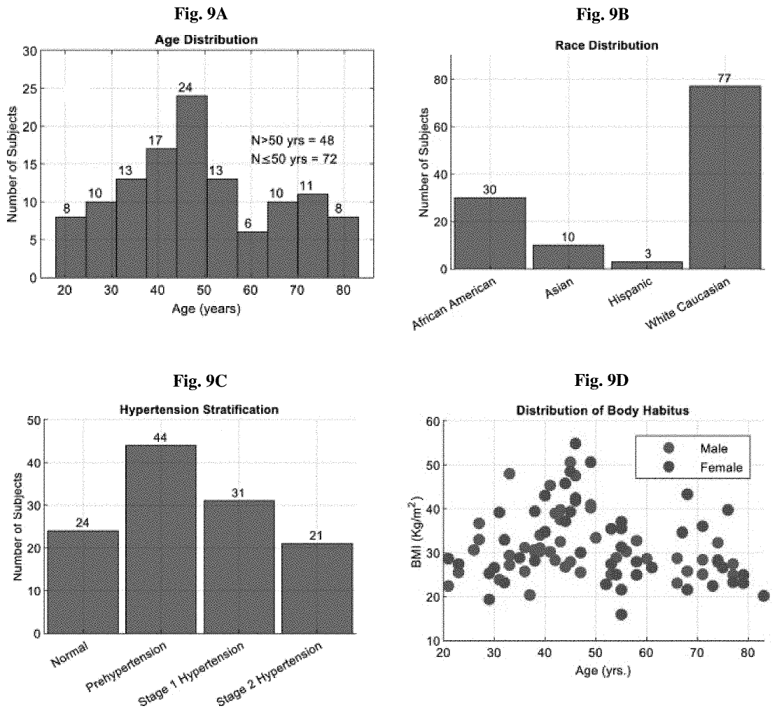

A total of 120 patients were recruited for the study. The recruited subject population was representative of the adult US census. The age distribution was 48.7±16.8 years, with 48% male. FIG. 9 presents (a) the histogram of age, (b) the distribution of race, (c) the hypertension stratifications as per Table 4 above, and (d) the distribution of age vs. Body Mass Index (BMI). The distribution of age vs. BMI shows the range of body habitus of the participating subjects. Body habitus is a metric that is necessary to analyze for wearable devices such as the SimpieSense™ device used in this paper.

### Blood Pressure Data

After the preparation of data collected in the study, the training set had 1348 observations of adequate quality, and the test set had 338 observations of adequate quality for use. FIG. 10 shows the distribution of the systolic and diastolic blood pressure values within the data sets.

### Feature Selection

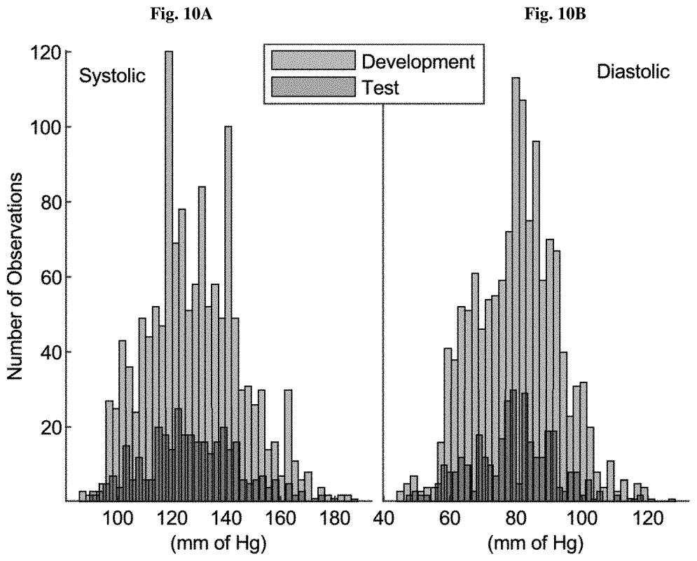

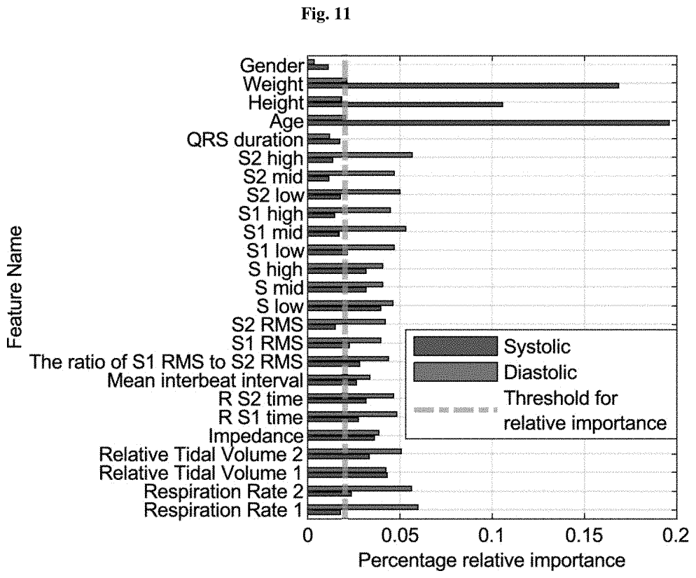

A random forest regression model was trained on the complete list of features in the training dataset for systolic blood pressure and diastolic blood pressure. Impurity-based feature importance was used to compute feature importance. Features that had more than 2% overall relative importance were selected. FIG. 11 shows the list of features and their importance for systolic blood pressure and diastolic blood pressure. The list of features that were considered important for systolic blood pressure were—Respiration rate 2, Relative Tidal Volume 1. Relative Tidal Volume 2, impedance, R to S1 time, R to S2 time, Mean interbeat interval, the ratio of S1 RMS to S2 RMS, S low, S mid, S high, QRS duration, age, height, and weight.

Similarly, the list of features that were considered important for diastolic blood pressure Respiration rate 2, Relative Tidal Volume 1. Relative Tidal Volume 2, impedance, R to S2 time, Mean interbeat interval, the ratio of S1 RMS to S2 RMS, S1 RMS, S low, S mid, S high, S1 low, age, height, and weight. Based on these feature selections, it is evident that demographic data is a strong predictor of systolic blood pressure and diastolic blood pressure values. However, there is potential benefit in terms of accuracy improvements resulting from using the SimpleSense™ measured signals in addition to demographics and machine learning methods to find the association with a hemodynarnic parameter that can be used to estimate blood pressure.

### Blood Pressure Model Descriptions

### Systolic Model

TABLE 5
Systolic Blood Pressure Estimation Ensemble of regression models

| Rank | Weight | Model Type | Model Parameters - (sklearn regressor) |
|---|---|---|---|
| 1 | 0.78 | Gradient Boosting | Loss function to be optimized = least squares |
| | | | Learning rate shrinks the contribution of each tree by |
| | | | learning_rate = 0.22526525740632317 |
| | | | The number of boosting stages to perform = 100 |
| | | | The fraction of samples to be used for fitting the individual base |
| | | | learners = 1.0 |
| | | | The function to measure the quality of a split = friedman_mse |
| | | | The minimum number of samples required to split an internal |
| | | | node = 8 |
| | | | The minimum number of samples required to be at a leaf node = 17 |
| | | | The minimum weighted fraction of the sum total of weights (of all |
| | | | the input samples) required to be at a leaf node = 0.0. |
| | | | Maximum depth of the individual regression estimators. The |
| | | | maximum depth limits the number of nodes in the tree = None. |
| | | | A node will be split if this split induces a decrease of the impurity |
| | | | greater than or equal to this value = 0.0. |
| | | | An estimator object that is used to compute the initial |
| | | | predictions = None. |
| | | | The fraction of total number of features to consider when looking |
| | | | for the best split = 0.7683292269455733 |
| | | | Grow trees with max_leaf_nodes in best-first fashion = None. |
| | | | Do not reuse the solution of the previous call to fit and add more |
| | | | estimators to the ensemble |
| | | | The proportion of training data to set aside as validation set for |
| | | | early stopping = 0.35101002117752517. |
| | | | n_iter_no_change is used to decide if early stopping will be used |
| | | | to terminate training when validation score is not improving = None |
| | | | Tolerance for the early stopping = 1e−07. |
| | | | Complexity parameter used for Minimal Cost-Complexity |
| | | | Pruning = 0.0. |
| 2 | 0.1 | K nearest neighbors | Number of neighbors to use by default = 1 |
| | | | Weight function used in prediction = ‘uniform’. |
| | | | Algorithm used to compute the nearest neighbors = ‘auto’ |
| | | | Power parameter for the Minkowski metric = 1. |
| | | | The distance metric to use for the tree = ‘minkowski’ |
| 3 | 0.06 | Support vector machine | Specifies the kernel type to be used in the algorithm = ‘rbf’ |
| | | regressor | Degree of the polynomial kernel function (‘poly’) = 3 |
| | | | Kernel coefficient for ‘rbf’, ‘poly’ and |
| | | | ‘sigmoid’ = 0.20113065159176252 |
| | | | Tolerance for stopping criterion = 0.0360184306323898. |
| | | | Regularization parameter = 194.03096694114694. |
| | | | Epsilon in the epsilon-SVR model. It specifies the epsilon-tube |
| | | | within which no penalty is associated in the training loss function |
| | | | with points predicted within a distance epsilon from the actual |
| | | | value = 0.0010647224198079256. |
| | | | Whether to use the shrinking heuristic = True, use shrinkage. |
| | | | Specify the size of the kernel cache = 200 MB |
| | | | Hard limit on iterations within solver = −1 |
| 4 | 0.04 | Adaboost regressor | The base estimator from which the boosted ensemble is |
| | | | built = None. |
| | | | The maximum number of estimators at which boosting is |
| | | | terminated = 217. |
| | | | Weight applied to each regressor at each boosting |
| | | | iteration = 0.8974499672625733. |
| | | | The loss function to use when updating the weights after each |
| | | | boosting iteration = ‘exponential’. |
| 5 | 0.02 | Random forest | The number of trees in the forest = 100. |
| | | regressor | The function to measure the quality of a split = friedman_mse. |
| | | | The maximum depth of the tree = None. |
| | | | The minimum number of samples required to split an internal |
| | | | node = 20 |
| | | | The minimum number of samples required to be at a leaf node = 11 |
| | | | The minimum weighted fraction of the sum total of weights (of all |
| | | | the input samples) required to be at a leaf node = 0.0. |
| | | | The fraction of the number of features to consider when looking |
| | | | for the best split = 0.7640742005089622 |
| | | | Grow trees with max_leaf_nodes in best-first fashion = None. |
| | | | A node will be split if this split induces a decrease of the impurity |
| | | | greater than or equal to this value = 0.0. |
| | | | Whether bootstrap samples are used when building trees = True. |
| | | | Do not reuse the solution of the previous call to fit and add more |
| | | | estimators to the ensemble. |
| | | | Complexity parameter used for Minimal Cost-Complexity |
| | | | Pruning = 0.0 |
| | | | the number of samples to draw from X to train each base |
| | | | estimator = All samples |

### Diastolic Model

TABLE 6
Diastolic Blood Pressure Estimation Ensemble of regression models

| | | Model | |
|---|---|---|---|
| Rank | Weight | Type | Model Parameters - (sklearn regressor) |
| 1 | 0.22 | Gradient | Loss function to be optimized = least squares. |
| | | Boosting | Learning rate shrinks the contribution of each tree by learning_rate = |
| | | | 0.11877384799096732 |
| | | | The number of boosting stages to perform = 100 |
| | | | The fraction of samples to be used for fitting the individual base learners = 1.0. |
| | | | The function to measure the quality of a split = friedman_mse. |
| | | | The minimum number of samples required to split an internal node = 2 |
| | | | The minimum number of samples required to be at a leaf node = 10. |
| | | | The minimum weighted fraction of the sum total of weights (of all the input |
| | | | samples) required to be at a leaf node = 0.0. |
| | | | Maximum depth of the individual regression estimators. The maximum depth |
| | | | limits the number of nodes in the tree = None. |
| | | | A node will be split if this split induces a decrease of the impurity greater than or |
| | | | equal to this value = 0.0. |
| | | | An estimator object that is used to compute the initial predictions = None. |
| | | | The number of features to consider when looking for the best split = None |
| | | | Grow trees with max_leaf_nodes in best-first fashion = 43. |
| | | | Donot reuse the solution of the previous call to fit and add more estimators to the |
| | | | ensemble. |
| | | | The proportion of training data to set aside as validation set for early stopping = |
| | | | 0.2841571323040151. |
| | | | n_iter_no_change is used to decide if early stopping will be used to terminate |
| | | | training when validation score is not improving = 12. |
| | | | Tolerance for the early stopping = 1e−07. |
| | | | Complexity parameter used for Minimal Cost-Complexity Pruning = 0.0. |
| 2 | 0.2 | Gradient | Loss function to be optimized = least squares. |
| | | Boosting | Learning rate shrinks the contribution of each tree by learning_rate = |
| | | | 0.04857791763352136 |
| | | | The number of boosting stages to perform = 100 |
| | | | The fraction of samples to be used for fitting the individual base learners = 1.0. |
| | | | The function to measure the quality of a split = friedman_mse. |
| | | | The minimum number of samples required to split an internal node = 2 |
| | | | The minimum number of samples required to be at a leaf node = 7. |
| | | | The minimum weighted fraction of the sum total of weights (of all the input |
| | | | samples) required to be at a leaf node = 0.0. |
| | | | Maximum depth of the individual regression estimators. The maximum depth |
| | | | limits the number of nodes in the tree = None. |
| | | | A node will be split if this split induces a decrease of the impurity greater than or |
| | | | equal to this value = 0.0. |
| | | | An estimator object that is used to compute the initial predictions = None. |
| | | | The number of features to consider when looking for the best split = None. |
| | | | Grow trees with max_leaf_nodes in best-first fashion = 1799. |
| | | | Do not reuse the solution of the previous call to fit and add more estimators to |
| | | | the ensemble. |
| | | | The proportion of training data to set aside as validation set for early |
| | | | stopping = 0.1. |
| | | | n_iter_no_change is used to decide if early stopping will be used to terminate |
| | | | training when validation score is not improving = 7. |
| | | | Tolerance for the early stopping = 1e−07. |
| | | | Complexity parameter used for Minimal Cost-Complexity Pruning = 0.0. |
| 3 | 0.14 | Gradient | Loss function to be optimized = least squares. |
| | | Boosting | Learning rate shrinks the contribution of each tree by learning_rate = |
| | | | 0.046029669658888245 |
| | | | The number of boosting stages to perform = 100 |
| | | | The fraction of samples to be used for fitting the individual base learners = 1.0. |
| | | | The function to measure the quality of a split = friedman_mse. |
| | | | The minimum number of samples required to split an internal node = 2 |
| | | | The minimum number of samples required to be at a leaf node = 7. |
| | | | The minimum weighted fraction of the sum total of weights (of all the input |
| | | | samples) required to be at a leaf node = 0.0. |
| | | | Maximum depth of the individual regression estimators. The maximum depth |
| | | | limits the number of nodes in the tree = None. |
| | | | A node will be split if this split induces a decrease of the impurity greater than or |
| | | | equal to this value = 0.0. |
| | | | An estimator object that is used to compute the initial predictions = None. |
| | | | The number of features to consider when looking for the best split = None. |
| | | | Grow trees with max_leaf_nodes in best-first fashion = 27. |
| | | | Do not reuse the solution of the previous call to fit and add more estimators to |
| | | | the ensemble. |
| | | | The proportion of training data to set aside as validation set for early |
| | | | stopping = 0.1. |
| | | | n_iter_no_change is used to decide if early stopping will be used to terminate |
| | | | training when validation score is not improving = 18. |
| | | | Tolerance for the early stopping = 1e−07. |
| | | | Complexity parameter used for Minimal Cost-Complexity Pruning = 0.0. |
| 4 | 0.14 | Gradient | Loss function to be optimized = least squares. |
| | | Boosting | Learning rate shrinks the contribution of each tree by learning_rate = |
| | | | 0.16896088251156324 |
| | | | The number of boosting stages to perform = 100 |
| | | | The fraction of samples to be used for fitting the individual base learners = 1.0. |
| | | | The function to measure the quality of a split = friedman_mse. |
| | | | The minimum number of samples required to split an internal node = 2 |
| | | | The minimum number of samples required to be at a leaf node = 14. |
| | | | The minimum weighted fraction of the sum total of weights (of all the input |
| | | | samples) required to be at a leaf node = 0.0. |
| | | | Maximum depth of the individual regression estimators. The maximum depth |
| | | | limits the number of nodes in the tree = None. |
| | | | A node will be split if this split induces a decrease of the impurity greater than or |
| | | | equal to this value = 0.0. |
| | | | An estimator object that is used to compute the initial predictions = None. |
| | | | The number of features to consider when looking for the best split = None |
| | | | Grow trees with max_leaf_nodes in best-first fashion = 8. |
| | | | Do not reuse the solution of the previous call to fit and add more estimators to |
| | | | the ensemble. |
| | | | The proportion of training data to set aside as validation set for early |
| | | | stopping = 0.1. |
| | | | n_iter_no_change is used to decide if early stopping will be used to terminate |
| | | | training when validation score is not improving = None. |
| | | | Tolerance for the early stopping = 1e−07. |
| | | | Complexity parameter used for Minimal Cost-Complexity Pruning = 0.0. |
| 5 | 0.14 | K nearest | Number of neighbors to use by default = 5 |
| | | neighbors | Weight function used in prediction = ‘uniform’ |
| | | | Algorithm used to compute the nearest neighbors = ‘auto’ |
| | | | Power parameter for the Minkowski metric = 2. |
| | | | The distance metric to use for the tree = ‘minkowski’. |
| 6 | 0.08 | Gradient | Loss function to be optimized = least squares. |
| | | Boosting | Learning rate shrinks the contribution of each tree by learning_rate = |
| | | | 0.022613964943578323 |
| | | | The number of boosting stages to perform = 100 |
| | | | The fraction of samples to be used for fitting the individual base learners = 1.0 |
| | | | The function to measure the quality of a split = friedman_mse. |
| | | | The minimum number of samples required to split an internal node = 2 |
| | | | The minimum number of samples required to be at a leaf node = 7. |
| | | | The minimum weighted fraction of the sum total of weights (of all the input |
| | | | samples) required to be at a leaf node = 0.0. |
| | | | Maximum depth of the individual regression estimators. The maximum depth |
| | | | limits the number of nodes in the tree = None. |
| | | | A node will be split if this split induces a decrease of the impurity greater than or |
| | | | equal to this value = 0.0. |
| | | | An estimator object that is used to compute the initial predictions = None. |
| | | | The number of features to consider when looking for the best split = None |
| | | | Grow trees with max_leaf_nodes in best-first fashion = 24. |
| | | | Do not reuse the solution of the previous call to fit and add more estimators to |
| | | | the ensemble. |
| | | | The proportion of training data to set aside as validation set for early |
| | | | stopping = 0.1. |
| | | | n_iter_no_change is used to decide if early stopping will be used to terminate |
| | | | training when validation score is not improving = 18. |
| | | | Tolerance for the early stopping = 1e−07. |
| | | | Complexity parameter used for Minimal Cost-Complexity Pruning = 0.0. |
| 7 | 0.06 | Gradient | Loss function to be optimized = least squares. |
| | | Boosting | Learning rate shrinks the contribution of each tree by learning_rate = |
| | | | 0.03079762265599878 |
| | | | The number of boosting stages to perform = 100 |
| | | | The fraction of samples to be used for fitting the individual base learners = 1.0. |
| | | | The function to measure the quality of a split = friedman_mse. |
| | | | The minimum number of samples required to split an internal node = 2 |
| | | | The minimum number of samples required to be at a leaf node = 36. |
| | | | The minimum weighted fraction of the sum total of weights (of all the input |
| | | | samples) required to be at a leaf node = 0.0. |
| | | | Maximum depth of the individual regression estimators. The maximum depth |
| | | | limits the number of nodes in the tree = None. |
| | | | A node will be split if this split induces a decrease of the impurity greater than or |
| | | | equal to this value = 0.0. |
| | | | An estimator object that is used to compute the initial predictions = None. |
| | | | The number of features to consider when looking for the best split = None |
| | | | Grow trees with max_leaf_nodes in best-first fashion = 331. |
| | | | Do not reuse the solution of the previous call to fit and add more estimators to |
| | | | the ensemble. |
| | | | The proportion of training data to set aside as validation set for early stopping = |
| | | | 0.23608035974923702. |
| | | | n_iter_no_change is used to decide if early stopping will be used to terminate |
| | | | training when validation score is not improving = 9. |
| | | | Tolerance for the early stopping = 1e−07. |
| | | | Complexity parameter used for Minimal Cost-Complexity Pruning = 0.0. |
| 8 | 0.02 | Gradient | Loss function to be optimized = least squares. |
| | | Boosting | Learning rate shrinks the contribution of each tree by learning_rate = |
| | | | 0.17024124189533613 |
| | | | The number of boosting stages to perform = 100 |
| | | | The fraction of samples to be used for fitting the individual base learners = 1.0. |
| | | | The function to measure the quality of a split = friedman_mse. |
| | | | The minimum number of samples required to split an internal node = 2 |
| | | | The minimum number of samples required to be at a leaf node = 14. |
| | | | The minimum weighted fraction of the sum total of weights (of all the input |
| | | | samples) required to be at a leaf node = 0.0. |
| | | | Maximum depth of the individual regression estimators. The maximum depth |
| | | | limits the number of nodes in the tree = None. |
| | | | A node will be split if this split induces a decrease of the impurity greater than or |
| | | | equal to this value = 0.0. |
| | | | An estimator object that is used to compute the initial predictions = None. |
| | | | The number of features to consider when looking for the best split = None |
| | | | Grow trees with max_leaf_nodes in best-first fashion = 14. |
| | | | Do not reuse the solution of the previous call to fit and add more estimators to |
| | | | the ensemble. |
| | | | The proportion of training data to set aside as validation set for early |
| | | | stopping = 0.1. |
| | | | n_iter_no_change is used to decide if early stopping will be used to terminate |
| | | | training when validation score is not improving = 4. |
| | | | Tolerance for the early stopping = 1e−07. |
| | | | Complexity parameter used for Minimal Cost-Complexity Pruning = 0.0. |

### Performance Evaluation

The Systolic and Diastolic models are ensembles of regression trees. The ensemble model was trained using the auto-sklearn framework. (Feurer, M. et al., Efficient and robust automated machine learning, Adv. Neural Inf. Process. Syst., 28,2962-2970, (2015)). With an overall time for a task set to 4 minutes and individual evaluations set to 30 seconds, the final diastolic model included eight tree-based regression models, and the systolic model included five regression models.

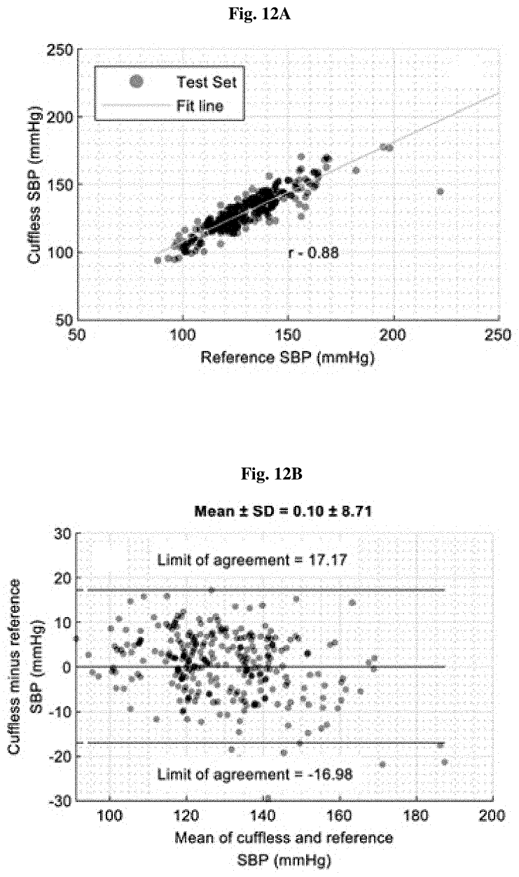

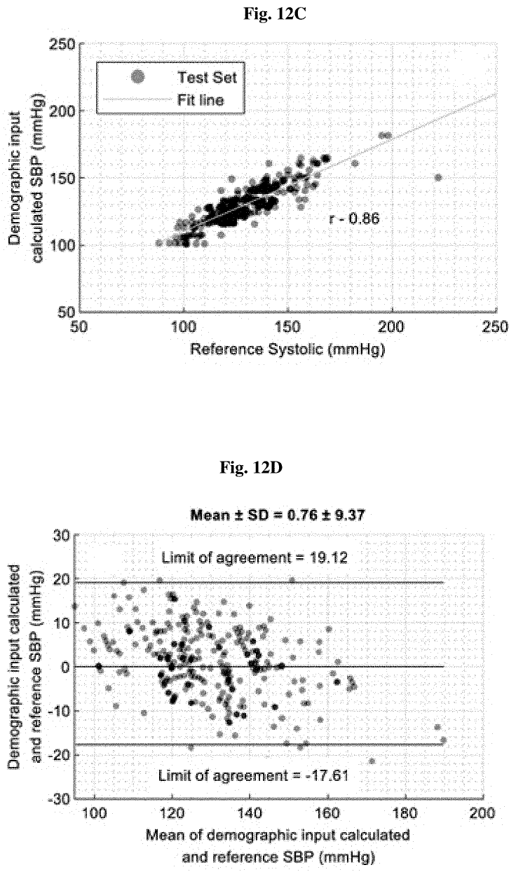

FIG. 12 illustrates the performance of the systolic blood pressure prediction models, The errors in prediction at the higher blood pressure values are greater at higher actual systolic blood pressures. The variability of blood pressure at higher systolic blood pressure is greater and leads to a biased estimate of performance when evaluated separately. (IEEE Standard for Wearable, Cuffless Blood Pressure Measuring Devices-Amendment 1, IEEE Std, 1708a-2019 (Amendment to IEEE Std 1708-2014) 1-35, (2019)). Additionally, age-related increases in variabilit of blood pressure are known. (Del Giorno, R., Balestra, L., Heiniger, P. S. & Ciabutti, L., Blood pressure variability with different measurement methods: Reliability and predictors. A proof of concept cross sectional study in elderly hypertensive hospitalized patients, Medicine. 98, e16347, (2019)).

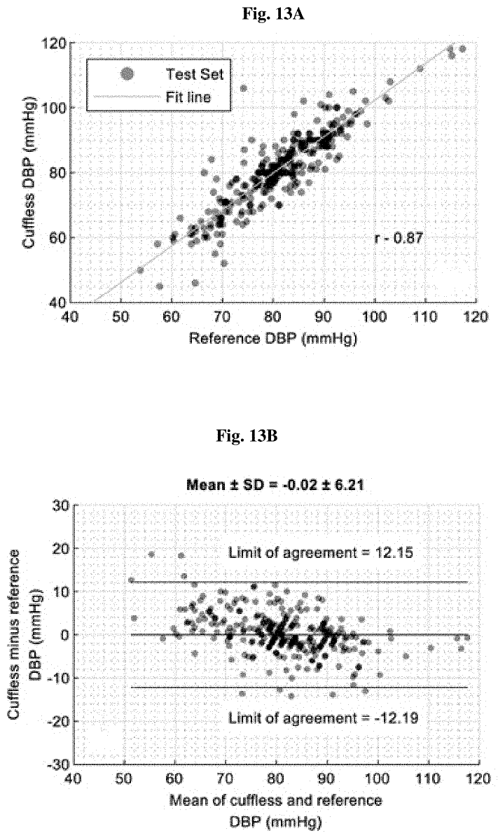

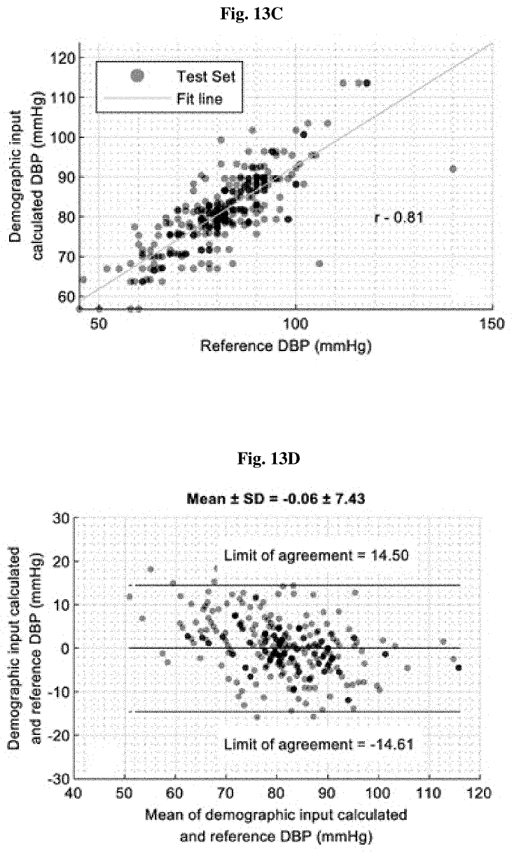

FIG. 13 illustrates the performance of the diastolic blood pressure prediction model. Notably, the d antic range of the observed diastolic blood pressure values is smaller than systolic blood pressure. The overestimation of diastolic blood pressure when the actual diastolic blood pressure is below 55 mm of Hg is observable. Although these errors may riot lead to a misinterpretation of prehypertensive in the individuals, additional sampling from those with low diastolic blood pressure values would improve the calibrated accuracy of the diastolic blood pressure models.

The results of bootstrap RMSE with the 95% CI are presented in Table 7 below.

TABLE 7
Results of the bootstrap RMSE analysis for each model
SimpleSense ™-BP, baseline model using demographics data only,
and the difference between the paired measurements of RMSE
between the models.

| Measure- | SimpleSense-BP | | SimpleSense- |
|---|---|---|---|
| ment | (RMSE- | Baseline model | BP-baseline model |
| type | mmHg) | (RMSE-mmHg) | (RMSE-mmHg) |
| SBP | 8.62 (6.87, 11.12) | 9.33 (7.82, 11.74) | −0.72 (−1.32, −0.14) |
| | | | (p < 0.0057) |
| DBP | 6.15 (4.87, 7.71) | 7.38 (6.19, 8.78) | −1.23 (−1.74, −0.74) |
| | | | (p < 0.0001) |

The test protocol includes induced variation of blood pressure within an individual participant using a cold pressor test, heating of extremities, relaxed. posture, and brisk exercise in the form of walking. Further, bootstrap resampling was done for the systolic blood pressure model and diastolic blood pressure model to obtain a 95% confidence interval for MAD values. 1000 iterations of resampling were performed with replacement with each sample size equal to the test set size and the confidence intervals for the systolic and diastolic MAD were found to be (5.36, 6.85 mm Hg) and (3.51, 4.65 mmHg), respectively.

### Discussion

Two ensemble models were trained on the data collected from 120 subjects. The two models predict systolic and diastolic pressures based on features extracted from the SimpleSense™ data and demographics data, inclusive of Age, Gender, Height, and Weight. This approach would be consistent with the class of blood pressure measurement called cuffless blood pressure monitors. The use of heart sounds, ECG, and thoracic impedance as inputs to estimate systolic blood pressure and diastolic blood pressure is novel Also. the size of the subject population for the evaluation of such a cuffless approach is the largest to date found in the literature. The device and model are cuffless and do not require a cuff calibration procedure. This removed the burden on the user to calibrate the device and eliminated errors due to the wrong calibration. The performance observed in this study supports a determination that this approach is indeed feasible for monitoring blood pressure of adults without a diagnosed arrhythmia or actively taking vasoactive anti-hypertensive medications. It is further observed that performance may improve at the low and high blood pressure ranges of measurement by increasing the number of subjects in those stratifications. However, as a generalized approach that should be reliable for the US population, the presented model meets the criteria for required performance as per the current IEEE 1708 standard. The present Example explored the possibility of including a broader population in terms of age, and the results are presented with age-stratified accuracy presented in Tables 8 and 9 of the supplementary materials below.

### Confounder Analysis Tables for Demographics Data that are Subject-Specific Systolic Model

TABLE 8
Confounder analysis table per IEEE 1708 2019a clause 4.6.2 for SBP

| Demographic | Number of | | MAD | MADP |
|---|---|---|---|---|
| Confounder | Measurements | Range | (mmHg) | (%) |
| ‘Age’ | 72 | ‘20.00-36.00’ | 4.43 | 3.60 |
| | 88 | ‘36.00-52.00’ | 6.64 | 4.87 |
| | 71 | ‘52.00-68.00’ | 5.12 | 4.16 |
| | | | 8.42 | |
| | 55 | ‘68.00-84.00’ | (value | 5.97 |
| | | | >7) | |

### Diastolic Model

TABLE 9
Confounder analysis table per IEEE 1708 2019a clause 4.6.2 for DBP

| Demographic | Number of | | MAD | MADP |
|---|---|---|---|---|
| Confounder | Measurements | Range | (mmHg) | (%) |
| ‘Age’ | 72 | ‘20.00-36.00’ | 3.45 | 4.55 |
| | 88 | ‘36.00-52.00’ | 4.43 | 5.01 |
| | 71 | ‘52.66-68.66’ | 3.64 | 4.98 |
| | 55 | ‘68.00-84.00’ | 4.67 | 6.34 |

Notably, it was observed that the age group of ≥68 years had an error rate for systolic blood pressure of 8.42 mmHg MAD. The subjects in this age range were 19 (15.83% of the population). Therefore, a larger number of subjects in this age range is warranted to improve the performance of SimpleSense™-BP.

The blood pressure estimation method presented herein as not developed to monitor blood pressure in ambulatory patients while performing activities involving movements. SimpleSense™ is wearable, and estimates of blood pressure can be calculated during movements. However, since the gold standard sphygmomanometer and existing ABPM devices do not claim blood pressure during movements, the training and testing data were collected with the subjects in a stationary state. Similarly, the current research that is presented emphasizes the algorithm that is developed to predict blood pressure. The algorithm was trained and validated based on the data presented in this article.

### Conclusion

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

Obvious variants of the disclosed embodiments are within the scope of the description and the claims that follow.

All references cited herein, as well as text appearing in the Figures and Tables, are hereby incorporated by reference in their entirety for all purposes to the same extent as if each were so individually denoted.

1-33. (canceled)

34. A method for predicting, estimating, and/or displaying blood pressure comprising:
a. selectively obtaining a plurality of input data from one or more measurement devices, selection and collection methods and/or processes;
b. conditioning the input data collected in step a using one or more data conditioning methods and processes;
c. Conducting feature extraction on the conditioned data of step b using one or more feature extraction methods and processes to extract one or more extracted features for blood pressure prediction;
d. conducting feature selection on the feature extracted data of step c using one or more feature selection methods and processes to select one or more selected features for blood pressure prediction; and
e. obtaining output which predicts, estimates and/or displays blood pressure from the feature selected data of step d by converting the feature selected data into predicted blood pressure values using one or more methods or processes selected from the group consisting of normalization, combination, transformation and combinations thereof.

35. The method of claim 34, wherein the input data is selected from the group consisting of input data and/or derivatives from electrical activity based metrics, input data and/or derivatives from bioimpedance based metrics, input data and/or derivatives from mechanical action metrics including goniometric measurements of all joint angles, absolute inclination, and orientation of different parts of the body in multiple axes, photoplethysmographic measures at various sites on the body such as fingers, toes, ankle, foot, arms, thorax, neck, and forehead, input data and/or derivatives from sounds including heart sounds, lung sounds, gastrointestinal sounds, and joint sounds, input data and/or derivatives from blood oxygen levels, data or derivatives from skin or body temperatures measured at different locations of the body including extremities, thorax, abdomen, and head, input data and/or derivatives from input data that can modulate pressure, and sweat biomarkers measured at various areas of the body.

36. The method of claim 34, wherein the predicted, estimated and/or displayed blood pressure values are converted into a graph representing a time-wise trend of the blood pressure values.

37. The method of claim 34, wherein the predicted, estimated and/or displayed blood pressure values are converted into a display of numeric systolic and diastolic values.

38. The method of claim 34, wherein the predicted, estimated and/or displayed blood pressure values are compared with a previously measured or previously predicted blood pressure values to provide an assessment on the increase or decrease of blood pressure over a period of time.

39. The method of claim 34, wherein the predicted, estimated and/or displayed blood pressure values include the use of a previously measured value, selected from the group consisting of peripheral vascular resistance of the patient, coronary resistance or the patient, arterial stiffness of the patient, aortic blood pressure of the patient, aortic blood pressure of the patient, left ventricular end diastolic pressures, pulmonary artery and venous pressures, measurements of chest circumference around the bottom of the sternum and combinations thereof.

40. The method of claim 34, wherein the method for predicting, estimating, and/or displaying blood pressure includes alarms or notifications in case of rapid blood pressure changes.

41. The method of claim 34, wherein the method for predicting, estimating, and/or displaying blood pressure provides a degree of confidence for each prediction, estimation and/or display of blood pressure in a range of from about 75% to about 95%.

42. A method for defining and processing data to provide model inputs to a blood pressure prediction model comprising the steps of:
a. Obtaining input data selected from the group consisting of input data and/or derivatives from electrical activity based metrics, input data and/or derivatives from bioimpedance based metrics, input data and/or derivatives from mechanical action metrics including goniometric measurements of all joint angles, absolute inclination, and orientation of different parts of the body in multiple axes, photoplethysmographic measures at various sites on the body such as fingers, toes, ankle, foot, arms, thorax, neck, and forehead, input data and/or derivatives from sounds including heart sounds, lung sounds, gastrointestinal sounds, and joint sounds, input data and/or derivatives from blood oxygen levels, input data and/or derivatives from skin or body temperatures measured at different locations of the body including extremities, thorax, abdomen, and head, input data and/or derivatives from input data that can modulate pressure, and sweat biomarkers measured at various areas of the body such as lactate, pH, alcohol, nicotine, sodium, glucose, urea, chloride, discrete blood, interstitial fluid, cerebrospinal fluid (CSF) biomarker panels, and metabolic panels, input data and/or derivatives from geographic location and altitude metrics, input data and/or derivatives from patient historic data and combinations thereof;
b. conditioning the input data of step a using a reversible transformation selected from the group consisting of filtering in time, frequency, wavelet, or other domains defined by a span of output of a convolutional neural network prior to a final layer which is a fully connected layer;
c. processing the conditioned input data of step b to obtain model inputs to the blood pressure prediction model using a method selected from the group consisting of converting the input data in time-series, subjecting the input data to feature extraction, computation, and transformation as predefined waveform patterns or time-varying quantities; processing the input data with unsupervised learning methods, processing the input data with regression-based methods, processing the input data with a trained neural network to achieve feature extraction, transforming the input data into an image or higher or lower dimensional space using reversible transformations and combinations thereof; and
d. inputting the processed data of step c as model inputs into the blood pressure prediction model.

43. The method of claim 34, wherein the feature extraction conducted is selected from the group consisting of rule-based extraction of waveform features from time series physiological data, using the output clusters of an unsupervised clustering method applied on input data at different granularities of time, applying the input data to a trained neural network for an alternative application such as image classification and using the output of a penultimate or earlier layer, using the output of the discriminator component of a generative adversarial network and combinations thereof.

44. The method of claim 42, wherein the method further comprises a signal and model assessment method comprising:
i) using a correction method to remove data collected by a measurement device used in a manner that does not produce a correct measurement, wherein the correction method accounts for data quality and confounders and is selected from the group consisting of thresholding techniques for level of movement, adaptive filtering techniques for remediation and combinations thereof.
ii) feature engineering the corrected data through an extraction process followed by a selection process comprising transformation and/or decomposition followed by feature selection.
iii) performing a process for the signal and model assessment to provide inputs for the blood pressure prediction model for improvements, conditioning, and correction, wherein the process is selected from the group consisting of a normalization process, a combination process, a transformation process and combinations thereof;
iv) performing a process selected from improvement methods and processes, correction methods and processes and combinations thereof to account for data quality and confounders;
v) performing data conditioning methods and processes for data conditioning and preparation;
vi) performing feature extraction methods and processes to extract a plurality of features for signal and model assessment from a plurality of measurement devices and historic patient data obtained after step ii;
vii) performing normalization, combination, and transformation methods and processes for the signal and model assessment to provide inputs for the blood pressure prediction model for improvements, conditioning, and correction.

45. The method of claim 42, further comprising a personalization method comprising:
a. performing one or more improvement, conditioning, and/or correction methods or processes to account for data quality and confounders;
b. performing data conditioning methods and processes for data conditioning and preparation of the data;
c. performing one or more feature extraction methods or processes to extract a plurality of features for signal and model assessment from one or more measurement devices and historic patient data
d. performing normalization, combination, and/or transformation methods and processes for the signal and model assessment to provide inputs for the blood pressure prediction model for improvements, conditioning, and correction.

46. The method of claim 42 wherein the method further comprises a continuous improvement method comprising:
a. performing improvements, conditioning, and/or correction methods and processes to account for data quality and confounders;
b. performing feature extraction methods and processes to extract a plurality of features for signal and model assessment from a plurality of measurement devices and historic patient data;
c. performing a plurality of feature selection methods and processes for selecting features that are relevant to blood pressure; and
d. performing one or more normalization, combination and/or transformation methods or processes for the signal and model assessment to provide inputs for the blood pressure prediction model for improvements, conditioning, and correction.

47. The method of claim 42, wherein a pre-trained model on a population is further trained with additional input data from an individual to generate a blood pressure prediction model that is unique for that individual, wherein the population.

48. The method of claim 42, wherein, additional data is obtained through a continuous blood pressure measurement technique for a time period extending from about a minute up to about forty five days by measuring the blood pressure through a known technique, wherein the blood pressures measured is used as a personalized training set for an individual to generate a blood pressure prediction model that is unique for an individual.

49. The method of claim 42, wherein the method predicts a degree of a confidence for each of the predicted outcomes associated therewith, each of the degrees of confidence based at least on the predicted data or the historical data regarding signal and model assessment methods and processes on a plurality of patients.

50. The method of claim 42, wherein a generative neural network includes a generator component and a discriminator component.

51. The method and process of claim 50, wherein, the input data applied to the discriminator generates a set of features is used to train another neural network or machine learning model to predict blood pressure.

52. The method of claim 44, wherein the techniques for level of movement are selected from the group consisting of an amplitude threshold, a frequency content threshold, an adaptive thresholds and combinations thereof.

53. The method of claim 44, wherein the adaptive filtering technique for remediation is recursive least squares filtering.

54. The method of claim 44, wherein the method of feature selection is selected from the group consisting of Principal Component Analysis to perform transformations to the features, eigen value to perform transformations to the features, vector decomposition to perform transformations to the features, box cox transformations to perform transformations to the features, measurement of mutual information using Kullback-Leibler convergence to perform regression to the features, minimum redundancy maximum relevance to perform regression to the features, impurity-based feature importance using random forest regression models to perform regression to the features, F-statistic or f-test to perform regression to the features, neighborhood component analysis to perform regression to the features, backward elimination to perform regression to the features, forward selection to perform regression to the features, permutation feature importance to perform regression to the features, factor analysis to perform regression to the features, and relief algorithm to perform regression to the features and combinations thereof.

55. The method of claim 54, wherein the feature selection is selected from the group consisting of minimum redundancy maximum relevance, impurity-based feature importance using random forest regression models and combinations thereof. c) a normalization, combination, or transformation process for the signal and model assessment to provide inputs for the blood pressure prediction model for improvements, conditioning, and correction.

56. The method of claim 44, wherein the normalization processes are selected from the group consisting of standard score, student's t statistic, studentized residual, standardized moment, min-max feature scaling and combinations thereof, the combination process is selected from the group consisting of support vector machines, linear regression, bagged trees, gradient boosting trees, extreme gradient boosting trees, Adaboost trees, random forests, k-nearest neighbors, gaussian process regression or other kernel-based regression techniques, multilayer perception neural networks, recurrent neural networks, convolution neural networks and combinations thereof and the transformation process is selected from the group consisting of scaling, weighted averaging, logistic regression probabilities and combinations thereof.

57. The method of claim 44, wherein the normalization process is min-max feature scaling, the combination process is gradient boosting trees, and the transformation process is scaling.

58. The method of claim 35, wherein the input data and/or derivatives from input data that can modulate pressure and sweat biomarkers measured at various areas of the body are selected from the group consisting of lactate, pH, alcohol, nicotine, sodium, glucose, urea, chloride, discrete blood, interstitial fluid, cerebrospinal fluid (CSF) biomarker panels, and metabolic panels, data or derivatives from geographic location and altitude metrics, data or derivatives from patient historic data and combinations thereof.

59. The method of claim 35, wherein the input data and/or derivatives from input data from mechanical metrics are selected from the group consisting of goniometric measurements of all joint angles, absolute inclination, and orientation of different parts of the body in multiple axes, photoplethysmographic measures at various sites on the body and combinations thereof.

60. The method of claim 35, wherein the input data and/or derivatives from input data from sounds is selected from the group consisting of including heart sounds, lung sounds, gastrointestinal sounds, joint sounds and combinations thereof.

61. The method of claim 35, wherein the input data and/or derivatives from input data from skin or body temperatures measured at different locations of the body is selected from the group consisting of extremities, thorax, abdomen, and head and combinations thereof.

62. The method of claim 35, wherein the sweat biomarkers are selected from the group consisting of lactate, pH, alcohol, nicotine, sodium, glucose, urea, chloride, discrete blood, interstitial fluid, cerebrospinal fluid (CSF) biomarker panels, metabolic panels and combinations thereof.

63. The method of claim 39, wherein the previously measured values are input data available from tests selected from the group consisting of echocardiographic imaging, measurements from a catheterization procedure, prescription of specific types of medications that affect the blood vessels such as vasodilators or vasoconstrictors and combinations thereof.
